(19) United States
(12) Patent Application Publication
(10) Pub. No.: US 2017/0312226 A1
(43) Pub. Date: Nov. 2, 2017

(54) PHARMACEUTICAL DOSAGE FORMS

(71) Applicant: Ascent Pharmaceuticals, Inc., Hauppauge, NY (US)

(72) Inventors: Sridhar Gumudavelli, Hauppauge, NY (US); Praneeth Rao Kakullamarri, Ronkonkoma, NY (US); Sudhakar Vidiyala, Hauppauge, NY (US); Madhava Reddy, Hauppauge, NY (US)

(21) Appl. No.: 15/140,844

(22) Filed: Apr. 28, 2016

(51) Int. Cl. A61K 9/20; A61K 9/48; A61K 9/28; A61K 9/24; A61K 31/485

(52) U.S. Cl. CPC A61K 9/2031; A61K 31/485; A61K 9/4866; A61K 9/4858; A61K 9/4833; A61K 9/4825; A61K 9/2009; A61K 9/209; A61K 9/2068; A61K 9/2054; A61K 9/2027; A61K 9/2013; A61K 9/2866

(57) ABSTRACT

This invention relates to pharmaceutical dosage forms for delivery of drugs susceptible to abuse, such as, for example, oxycodone and/or oxymorphone.

### BACKGROUND OF THE INVENTION

This invention relates to pharmaceutical dosage forms for delivery of drugs susceptible to abuse, such as, for example, oxycodone and/or oxymorphone.

In certain embodiments, the present invention relates to Self-Emulsified Abuse and Tamper Resistant Liquid filled dosage forms. In certain embodiments, the present invention relates to Abuse and Tamper Resistant Solid Dosage Forms containing Therapeutic Agent in complex form with ion-exchange resin. In certain embodiments, the present invention relates to Abuse and Tamper Resistant Solid Dosage forms containing Therapeutic agent in microencapsulated form.

In certain embodiments, the present invention relates to self-emulsified semi-solid high viscosus liquids that formulate into hard gelatin and/or soft gelatin capsules having abuse and tamper resistant properties. Abuse and tamper resistant formulations comprise one or more therapeutic active moieties in molecular form with one or more high and/or low HLB surfactants and co-surfactants, one or more triglycerides and a pH independent polymer ethylcellulose.

In additional embodiments, the present invention relates to controlled release abuse and tamper resistant tablets comprising at least one drug susceptible to abuse, such as an opioid, for example oxycodone HCl, wherein the tablet comprises a resin complex of the drug susceptible to abuse imbibed in a mixture of a swellable, and non-swellable hydrophilic, non-swellable lipophilic and wax polymeric matrix.

In further embodiments, the present invention discloses a novel dosage form for delivering at least one drug susceptible to abuse. Further, it releases drug in a controlled rate over a period of time at a variable rate across the pH range from 1.0 to 8.0. The therapeutic agent and agents may be present in ion exchange form. The osmotic drug delivery system includes a gelling agent with one or more salts and inorganic acids to provide a particular viscosity, and enhances the solubility power of the hydrated core in order to release the therapeutic agent in molecular form through an orifice at a predefined release rate. Additionally, in the present invention at least one therapeutic agent may be present in an ion exchange complex form; this renders abuse-capable matrix system with one or more gelling agents that are difficult to separate the opioid and other drugs prone to abuse from the finished drug dosage form. The osmotic drug delivery system provides a therapeutically effective steady state plasma concentration for 12 to 24 hours when administered twice or once per day.

In other embodiments, the present invention relates to abuse and tamper resistant controlled release solid dosage form containing at least one therapeutic agent susceptible to abuse in an ion exchange resin complex form. Furthermore, the present invention contains, for example, low molecular hydrophilic polyethylene oxide, a water soluble ionic compound, and a non-digestible wax.

The present invention further discloses an abuse and tamper resistant lipophilic drug delivery system predominantly administered orally and encompassing at least one opioid active prone to substance abuse by alcoholic extraction and/or other tampering methods including crushing, and grinding. Furthermore, the invention involves a phenomenon most commonly referred to as “Sintering”, wherein an active agent adheres to an adjacent lipophilic surface in a mass of powder and/or in a compact by heat and/or compression and/or compression followed by heat. The said abuse and tamper resistant dosage form comprises one or more functional excipients in an effective amount to make a dosage form unsuitable for administration of parenteral and nasal administration. The preparation involves granulation of an opioid active using a mixture of hydrophilic and lipophilic binders in a solid or liquid state at regular or elevated temperatures to form granules that modulate the release of the active ingredient for abuse intended extraction along with lubrication and coating agents.

All inventions herein can formulated into immediate or extended/controlled release dosage forms by modulating one and/or more functional excipients such as, for example, xanthan gum, fatty acids, waxes, surfactants and co-surfactants.

All references cited herein are incorporated herein by reference in their entireties.

### BRIEF SUMMARY OF THE INVENTION

The invention provides an lipophilic abuse and tamper resistant drug delivery system comprising: i) at least one active agent susceptible to abuse selected from the group consisting of opiates, opioids, tranquilizers, stimulants, narcotics, and combinations thereof; ii) optionally, at least one ion exchange resin; iii) at least one binder for granulation; iv) optionally at least one surfactant; v) optionally at least one wax; vi) optionally, at least one synthetic or natural polymer; vii) optionally at least one excipients; and viii) optionally at least one viscosity enhancing agent. The invention further provides a drug delivery system which is in the form of a tablet. The invention further provides a drug delivery system which is in the form of a coated tablet. The invention further provides a drug delivery system which is in the form of an uncoated tablet. The invention further provides a drug delivery system which is in multiparticulate form. The invention further provides a drug delivery system wherein the at least one active agent susceptible to abuse is present in an amount of about 10 to about 60 wt % of the composition. The invention further provides a drug delivery system wherein the at least one ion exchange resin is present in an amount of about 10 to about 30 wt % of the composition. The invention further provides a drug delivery system wherein the ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof. The invention further provides a drug delivery system wherein the at least one binder for granulation is present in an amount of about 10 to about 40 wt % of the composition.

The invention further provides a drug delivery system wherein the at least binder for granulation is present and is selected from the group consisting of natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, carnauba wax, glycerol behenate (COMPRITOL® 888 ATO), glycerylmonostereate, glycerol palmitostearate (PRECIROL®), and hydrophilic substances selected from a group of water soluble or water insoluble, non-gelling binders, Poly(vinyl) pyrrolidone, Poly(vinyl) alcohol, starch, corn starch, pregelatinized starch, microcrystalline cellulose (MCC), silicified MCC, microfine cellulose, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropylmethyl celluloses, and combinations thereof.

The invention further provides a drug delivery system wherein the at least one surfactant is present in an amount of about 2 to about 30 wt % of the composition. The invention further provides a drug delivery system wherein the at least one wax is present in an amount of about 2 to about 30 wt % of the composition. The invention further provides a drug delivery system wherein the at least one wax is selected from the group consisting of carnauba wax, white wax, natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, carnauba wax, and combinations thereof. The invention further provides a drug delivery system wherein the at least one synthetic or natural polymer is present in an amount of about 0 to about 20 wt % of the composition. The invention further provides a dosage form wherein the at least one synthetic or natural polymer is at least one polymer selected from the group consisting of polyethylene oxide, polymethylene oxide, polypropylene oxide, polyethylene, polypropylene, polyvinyl chloride, polycarbonate, polystyrene, polyacrylate, copolymers and mixtures thereof. The invention further provides a drug delivery system wherein the at least one excipients is present in an amount of about 10 to about 50 wt % of the composition. The invention further provides a drug delivery system wherein the at least one viscosity enhancing agent is present in an amount of about 10 to about 20 wt % of the composition.

The invention further provides a dosage form wherein the at least one viscosity-increasing agent is selected from the group consisting of microcrystalline cellulose with 11 wt. % carboxymethylcellulose sodium (Avice® RC 591), carboxymethylcellulose sodium (Blanose®, CMC-Na C300P®, Frimulsion BLC-5®, Tylose C300 P®), polyacrylic acid (Carbopol® 980 NF, Carbopol® 981), locust bean flour (Cesagum® LA-200, Cesagum® LID/150, Cesagum® LN-1), citrus pectin (Cesapectin® HM Medium Rapid Set), waxy maize starch (C*Gel 04201®), sodium alginate (Frimulsion ALG (E401)®), guar flour (Frimulsion BM®, Polygum 26/1-75®), iota carrageen (Frimulsion D021®), karaya gum, gellan gum (Kelcogel F®, Kelcogel LT100®), galactomannan (Meyprogat 150®), tara bean flour (Polygum 43/1®), propylene glycol alginate (Protanal-Ester SD-LB®), sodium hyaluronate, apple pectin, pectin from lemon peel, sodium hyaluronate, tragacanth, tara gum (Vidogum SP 200®), fermented polysaccharide welan gum (K1A96) and xanthan gum (Xantural 180®).

The invention further provides a dosage form wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, melazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof. The invention further provides a dosage form wherein the dosage form has a breaking strength of at least 1000 N. The invention further provides a dosage form which comprises at least one active ingredient at least partially in controlled release form. The invention further provides a dosage form wherein the at least one active agent susceptible to abuse is present in a controlled release matrix.

The invention provides a process for the production of a dosage form comprising:

mixing components: i) at least one active agent susceptible to abuse; ii) optionally, at least one ion exchange resin; iii) at least one binder for granulation; iv) optionally at least one surfactant; v) optionally at least one wax; vi) optionally, at least one synthetic or natural polymer; vii) optionally at least one excipients; and viii) optionally at least one viscosity enhancing agent, to form a resultant mixture, and press-forming the resultant mixture, optionally after granulation, to yield the dosage form with preceding, simultaneous, or subsequent exposure to heat. The invention further provides a process wherein granulation is performed by means of a melt process. The invention further provides a process which comprises press-forming the resultant mixture to yield a press-formed product, and exposing the press-formed product to heat to yield the dosage form. The invention further provides a dosage form obtainable by the process. The invention provides an oral abuse and tamper resistant pharmaceutical solid dosage form comprising: i) at least one active agent susceptible to abuse selected from the group consisting of opiates, opioids, tranquilizers, stimulants and narcotics; ii) at least one surfactant, wherein the surfactant is selected from the group consisting of high hydrophilic/lipophilic balance (HLB) surfactants, low HLB surfactants, and a combination thereof; iii) at least one ethylcellulose polymer, wherein the ethylcellulose polymer is selected from the group consisting of high viscosity ethylcellulose polymer, low viscosity ethylcellulose polymer, and a combination thereof; iv) oleic acid; and v) at least one hydrophilic solvent, wherein said oral abuse and tamper resistant pharmaceutical solid dosage is in unit dosage form.

The invention further provides an oral abuse and tamper resistant pharmaceutical solid dosage form wherein the at least one active agent susceptible to abuse is present in an amount of about 10 to about 60 wt % of the composition. The invention further provides an oral abuse and tamper resistant pharmaceutical solid dosage form wherein the at least one surfactant is present in an amount of about 2 to about 30 wt % of the composition. The invention further provides an oral abuse and tamper resistant pharmaceutical solid dosage form wherein the polymer is present in an amount of about 0 to about 20 wt % of the composition. The invention further provides an oral abuse and tamper resistant pharmaceutical solid dosage form wherein the oleic acid is present in an amount of 10 to 70 wt % of the composition. The invention further provides an oral abuse and tamper resistant pharmaceutical solid dosage form wherein the at least one hydrophilic solvent is present in an amount of about 5 to about 30 wt % of the composition. The invention further provides an oral abuse and tamper resistant pharmaceutical solid dosage form wherein the at least one hydrophilic solvent is selected from the group consisting of water, ethanol, glycerol, glycols, polyols, and combinations thereof. The invention further provides an oral abuse and tamper resistant pharmaceutical solid dosage form wherein the composition is immediate release. The invention further provides an oral abuse and tamper resistant pharmaceutical solid dosage form wherein the composition is delayed release.

The invention further provides an oral abuse and tamper resistant pharmaceutical solid dosage form wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof. The invention further provides an oral abuse and tamper resistant pharmaceutical solid dosage form wherein the at least one active agent susceptible to abuse comprises oxymorphone or oxycodone.

The invention provides an oral abuse and tamper resistant pharmaceutical solid dosage form comprising: i) oxycodone present in an amount of about 7 to about 9 wt % of the composition; ii) ethylcellulose present in an amount of about 9 to about 11 wt % of the composition; iii) oleic acid present in an amount of about 50 to about 70 wt % of the composition; iv) PEG-8 caprylic/capric glycerides present in an amount of about 8 to about 12 wt % of the composition; and v) polyoxyl 40 hydrogenated castor oil present in an amount of about 8 to about 12 wt % of the composition.

The invention provides a capsule comprising the oral abuse and tamper resistant pharmaceutical solid dosage form. The invention provides a capsule wherein the capsule is a hard gelatin capsule or a soft gelatin capsule.

The invention provides a method of making oral abuse and tamper resistant pharmaceutical solid dosage form comprising a tamper resistant controlled release matrix comprising: i) at least one active agent susceptible to abuse selected from the group consisting of opiates, opioids, tranquilizers, stimulants and narcotics; ii) at least one surfactant, wherein the surfactant is selected from the group consisting of high hydrophilic/lipophilic balance (HLB) surfactants, low HLB surfactants, and a combination thereof; iii) at least one ethylcellulose polymer, wherein the ethylcellulose polymer is selected from the group consisting of high viscosity ethylcellulose polymer, low viscosity ethylcellulose polymer, and a combination thereof; iv) oleic acid; and v) at least one hydrophilic solvent, wherein composition is in a unit dosage form, the method comprising: mixing the surfactant, ethylcellulose polymer, and oleic acid; adding the drug susceptible to abuse with continuous mixing; heating; and encapsulating, thereby making the oral abuse and tamper resistant pharmaceutical solid dosage form. The invention further provides a method wherein the at least one active agent susceptible to abuse is present in an amount of about 10 to about 60 wt % of the composition. The invention further provides a method wherein the at least one surfactant is present in an amount of about 2 to about 30 wt % of the composition. The invention further provides a method wherein the polymer is present in an amount of about 0 to about 20 wt % of the composition. The invention further provides a method wherein the oleic acid is present in an amount of 10 to 70 wt % of the composition. The invention further provides a method wherein the at least one hydrophilic solvent is present in an amount of about 5 to about 30 wt % of the composition. The invention further provides a method wherein the at least one hydrophilic solvent is selected from the group consisting of water, ethanol, glycerol, glycols, polyols, and combinations thereof. The invention further provides a method wherein the composition is immediate release. The invention further provides a method wherein the composition is delayed release.

The invention further provides a method wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof. The invention further provides a method wherein the at least one active agent susceptible to abuse comprises oxymorphone or oxycodone. The invention provides an oral abuse and tamper resistant pharmaceutical solid dosage form composition comprising a tamper resistant controlled release matrix, wherein the dosage form comprises: i) at least one active agent susceptible to abuse selected from the group consisting of opiates, opioids, tranquilizers, stimulants, narcotics, and combinations thereof; ii) at least one ion exchange resin; iii) at least one swellable polyethylene polymer; iv) at least one non-swellable low molecular weight polyethylene glycol; and iv) at least one hydrophobic binder, wherein said composition is in a unit dosage form. The invention further provides an oral abuse and tamper resistant pharmaceutical solid dosage form wherein the pharmaceutical composition is in tablet form. The invention further provides an oral abuse and tamper resistant pharmaceutical solid dosage form wherein the at least one active agent susceptible to abuse is present in an amount of about 10 to about 60 wt % of the composition. The invention further provides an oral abuse and tamper resistant pharmaceutical solid dosage form wherein the at least one ion exchange resin is present in an amount of about 10 to about 30 wt % of the composition.

The invention further provides an oral abuse and tamper resistant pharmaceutical solid dosage form wherein the at least one ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof. The invention further provides an oral abuse and tamper resistant pharmaceutical solid dosage form wherein the at least one swellable polyethylene polymer present in an amount of about 10 to about 60 wt % of the composition; The invention further provides an oral abuse and tamper resistant pharmaceutical solid dosage form wherein the at least one non-swellable low molecular weight polyethylene glycol present in an amount of about 10 to about 60 wt % of the composition; and The invention further provides an oral abuse and tamper resistant pharmaceutical solid dosage form wherein the at least one hydrophobic binder is present in an amount of about 10 to about 40 wt % of the composition.

The invention further provides an oral abuse and tamper resistant pharmaceutical solid dosage form wherein the at least one hydrophobic binder is selected from the group consisting of natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, and carnauba wax. The invention further provides an oral abuse and tamper resistant pharmaceutical solid dosage form wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof. The invention provides an oral abuse and tamper resistant pharmaceutical solid dosage form composition comprising a tamper resistant controlled release matrix, wherein the composition comprises: i) oxycodone HCl present in an amount of about 10 to about 60 wt % of the composition; ii) sodium polystyrene sulfonate present in an amount of about 10 to about 60 wt % of the composition; iii) polyethylene oxide present in an amount of about 10 to about 60 wt % of the composition; iv) polyethylene glycol present in an amount of about 10 to about 60 wt % of the composition; v) carnuba wax present in an amount of about 10 to about 60 wt % of the composition; vi) microcrystalline cellulose present in an amount of about 10 to about 60 wt % of the composition; vi) colloidal silicon dioxide present in an amount of about 10 to about 60 wt % of the composition; and vii) stearic acid present in an amount of about 10 to about 60 wt % of the composition. The invention provides a method of making an oral abuse and tamper resistant pharmaceutical solid dosage form comprising a tamper resistant controlled release matrix the method comprising the steps of: providing the ingredients: i) at least one active agent susceptible to abuse selected from the group consisting of opiates, opioids, tranquilizers, stimulants, narcotics, and combinations thereof; ii) at least one ion exchange resin; iii) at least one swellable polyethylene polymer; iv) at least one non-swellable low molecular weight polyethylene glycol; and iv) at least one hydrophobic binder, the method comprising: mixing all ingredients; passing through a 40 mesh screen; compressing into tablets; and curing at about 70° C. for about 200 seconds. The invention further provides a method wherein the pharmaceutical composition is in tablet form. The invention further provides a method wherein the at least one active agent susceptible to abuse is present in an amount of about 10 to about 60 wt % of the composition. The invention further provides a method wherein the at least one ion exchange resin is present in an amount of about 10 to about 30 wt % of the composition. The invention further provides a method wherein the at least one ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof. The invention further provides a method wherein the at least one swellable polyethylene polymer present in an amount of about 10 to about 60 wt % of the composition; The invention further provides a method wherein the at least one non-swellable low molecular weight polyethylene glycol present in an amount of about 10 to about 60 wt % of the composition; and The invention further provides a method wherein the at least one hydrophobic binder is present in an amount of about 10 to about 40 wt % of the composition. The invention further provides a method wherein the at least one hydrophobic binder is selected from the group consisting of natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, and carnauba wax. The invention further provides a method wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydromorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

The invention provides an oral abuse and tamper resistant pharmaceutical solid dosage form composition comprising granules in an extragranular matrix, wherein: the granules comprise: i) at least one active agent susceptible to abuse selected from the group consisting of opiates, opioids, tranquilizers, stimulants, narcotics, and combinations thereof; ii) optionally, at least one ion exchange resin; iii) at least one binder for granulation; iv) optionally at least one surfactant; v) optionally at least one wax; vi) optionally, at least one synthetic or natural polymer; vii) optionally at least one excipients, and the extragranular matrix comprises: viii) at least one synthetic or natural polymer ix) at least one gelling agent; x) at least one osmotic agent selected from the group consisting of salts and organic acids xi) optionally at least one viscosity enhancing agent, wherein the pharmaceutical composition is an osmotic controlled release dosage form. The invention further provides an oral abuse and tamper resistant pharmaceutical solid dosage form wherein the at least one active agent susceptible to abuse present in an amount of about 10 to about 60 wt % of the composition. The invention further provides an oral abuse and tamper resistant pharmaceutical solid dosage form wherein the at least one ion exchange resin is present in an amount of about 10% to about 30% of the composition.

The invention further provides an oral abuse and tamper resistant pharmaceutical solid dosage form wherein the ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof. The invention further provides an oral abuse and tamper resistant pharmaceutical solid dosage form wherein the at least one gelling agent is present in an amount of about 10 to about 60 wt % of the composition. The invention further provides an oral abuse and tamper resistant pharmaceutical solid dosage wherein the gelling agent in an effective amount to impart a viscosity unsuitable for parenteral administration when the dosage form is subjected to tampering wherein the gelling agent is selected from the group consisting of mannitol, sorbitol, starch, starch derivatives, cellulose derivatives, microcrystalline cellulose, sodium caboxymethyl cellulose, methylcellulose, ethyl cellulose, hydroxyethyl cellulose, hydroxypropyl cellulose, hydroxypropyl methylcellulose, attapulgites, bentonites, dextrins, alginates, carrageenan, gum tragacanth, gun acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and combinations thereof; The invention further provides an oral abuse and tamper resistant pharmaceutical solid dosage form wherein the at least one osmotic agent selected from the group consisting of salts and organic acids, present in an amount of about 10 to about 60 wt % of the composition.

The invention further provides an oral abuse and tamper resistant pharmaceutical solid dosage form wherein the controlled release dosage form is a bilayer osmotic controlled release dosage form. The invention further provides an oral abuse and tamper resistant pharmaceutical solid dosage form wherein said osmotic controlled release dosage form comprises a bilayer tablet comprising an orifice. The invention further provides an oral abuse and tamper resistant pharmaceutical solid dosage form wherein the controlled release dosage form is a matrix controlled release dosage form. The invention further provides an oral abuse and tamper resistant pharmaceutical solid dosage form wherein the at least one active agent susceptible to abuse is present in an amount of about 10 to about 60 wt % of the composition. The invention further provides an oral abuse and tamper resistant pharmaceutical solid dosage form wherein the pharmaceutical composition is in unit dose form. The invention further provides an oral abuse and tamper resistant pharmaceutical solid dosage form wherein the pharmaceutical composition is in tablet form. The invention further provides an oral abuse and tamper resistant pharmaceutical solid dosage form wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

The invention provides an oral abuse and tamper resistant pharmaceutical solid dosage form composition comprising a drug layer and a push layer, wherein the drug layer comprises: ii) oxycodone present in an amount of about 10 to about 60 wt % of the composition; ii) polyethylene oxide present in an amount of about 10 to about 60 wt % of the composition; iii) povidone present in an amount of about 10 to about 60 wt % of the composition; iv) succinic acid present in an amount of about 10 to about 60 wt % of the composition; v) magnesium stearate present in an amount of about 10 to about 60 wt % of the composition; vi) butylated hydroxytoluene present in an amount of about 10 to about 60 wt % of the composition; vii) isopropyl alcohol present in an amount of about 10 to about 60 wt % of the composition; and viii) water, q.s., further wherein the push layer comprises: i) polyethylene oxide present in an amount of about 10 to about 60 wt % of the composition; ii) sodium chloride in an amount of about 10 to about 60 wt % of the composition; iii) povidone present in an amount of about 10 to about 60 wt % of the composition; iv) magnesium stearate present in an amount of about 10 to about 60 wt % of the composition; v) butylated hydroxytoluene present in an amount of about 10 to about 60 wt % of the composition; vi) yellow iron oxide present in an amount of about 10 to about 60 wt % of the composition; vii) isopropyl alcohol present in an amount of about 10 to about 60 wt % of the composition; and viii) water, q.s.

The invention provides a method of making an oral abuse and tamper resistant pharmaceutical solid dosage form comprising granules in an extragranular matrix, the method comprising: providing the granule ingredients which comprise: i) at least one active agent susceptible to abuse selected from the group consisting of opiates, opioids, tranquilizers, stimulants, narcotics, and combinations thereof; ii) optionally, at least one ion exchange resin; iii) at least one binder for granulation; iv) optionally at least one surfactant; v) optionally at least one wax; vi) optionally, at least one synthetic or natural polymer; vii) optionally at least one excipients, mixing the granule ingredients; providing the extragranular matrix ingredients which comprise: viii) at least one synthetic or natural polymer; ix) at least one gelling agent; x) at least one osmotic agent selected from the group consisting of salts and organic acids; xi) optionally at least one viscosity enhancing agent, mixing the extragranular ingredients; combining the granular and extragranular ingredients; and making the osmotic controlled release dosage form. The invention further provides a method wherein the at least one active agent susceptible to abuse present in an amount of about 10 to about 60 wt % of the composition; The invention further provides a method wherein the at least one ion exchange resin is present in an amount of about 10% to about 30% of the composition. The invention further provides a method wherein the ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof. The invention further provides a method wherein the at least one gelling agent is present in an amount of about 10 to about 60 wt % of the composition.

The invention further provides a method wherein the gelling agent in an effective amount to impart a viscosity unsuitable for parenteral administration when the dosage form is subjected to tampering wherein the gelling agent is selected from the group consisting of mannitol, sorbitol, starch, starch derivatives, cellulose derivatives, microcrystalline cellulose, sodium caboxymethyl cellulose, methylcellulose, ethyl cellulose, hydroxyethyl cellulose, hydroxypropyl cellulose, hydroxypropyl methylcellulose, attapulgites, bentonites, dextrins, alginates, carrageenan, gum tragacanth, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and combinations thereof;

The invention further provides a method wherein the at least one osmotic agent selected from the group consisting of salts and organic acids, present in an amount of about 10 to about 60 wt % of the composition. The invention further provides a method wherein the controlled release dosage form is a bilayer osmotic controlled release dosage form. The invention further provides a method wherein said osmotic controlled release dosage form comprises a bilayer tablet comprising an orifice. The invention further provides a method wherein the controlled release dosage form is a matrix controlled release dosage form. The invention further provides a method wherein the at least one active agent susceptible to abuse is present in an amount of about 10 to about 60 wt % of the composition. The invention further provides a method wherein the pharmaceutical composition is in unit dose form. The invention further provides a method wherein the pharmaceutical composition is in tablet form.

The invention further provides a method wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

The invention provides an oral abuse and tamper resistant solid dosage form comprising, i) at least one active agent susceptible to abuse selected from the group consisting of opiates, opioids, tranquilizers, stimulants and narcotics present in complex form with one or more ion-exchange resin, low molecular weight polyethylene oxide; at least one water soluble ionic compound; at least one non-digestible wax; and a mixture of at least one a low melting point stearic acid and at least one low melting point palmitic acid. The invention further provides an oral abuse and tamper resistant solid dosage form wherein the active ingredient is selected from the group consisting of opiates, opioid, tranquilizers, stimulants, narcotics, alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof. The invention further provides an oral abuse and tamper resistant solid dosage form wherein the polyethylene oxide is present in an amount of about 10 to about 60 wt % of the composition. The invention further provides an oral abuse and tamper resistant solid dosage form wherein the polyethylene oxide molecular weight is below 200,000 and preferably 200,000. The invention further provides an oral abuse and tamper resistant solid dosage form wherein the water soluble ionic compounds are selected from the group consisting of sodium chloride, potassium chloride, and mixtures thereof. The invention further provides an oral abuse and tamper resistant solid dosage form wherein the non-digestible wax material is present in an amount of about 10 to about 60 wt % of the composition.

The invention further provides an oral abuse and tamper resistant solid dosage form wherein the non-digestible wax material is carnauba wax, white wax, natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax and carnauba wax. The invention further provides an oral abuse and tamper resistant solid dosage form wherein polyethylene oxide from about 5 to about 75 wt %. The invention further provides an oral abuse and tamper resistant solid dosage form wherein the ionic compound is present in an amount of from about 0.5% to about 30 wt % of the composition. The invention further provides an oral abuse and tamper resistant solid dosage form wherein the non-digestible wax is present in an amount of from about 2.5 to about 35 wt %.

The invention further provides a lipophilic abuse and tamper resistant drug delivery system that in its final form includes a tablet with or without a protective coat, formed by compressing granules produced by hot melt granulation of an opioid active that may or may not be complexed with an ion exchange resin, achieved through addition of binders added in solid or liquid state, either with or without suitable lubrication and viscosity enhancing agents. The invention further provides a lipophilic abuse and tamper resistant drug delivery system where in the final dosage form is obtained by compressing two or more granulation mixtures where in at least one layer will include granules produced by hot melt granulation of an opioid active that may or may not be complexed with an ion exchange resin, achieved through addition of binders added in solid or liquid state. The invention further provides a lipophilic abuse and tamper resistant drug delivery system wherein the release profile can be modulated by either varying several components and their levels or by modifying the method of manufacturing. The invention further provides a lipophilic abuse and tamper resistant drug delivery system wherein the variation of the components to alter the drug release includes modifying the concentration of drug release retarding agents in the formulation. The invention further provides a lipophilic abuse and tamper resistant drug delivery system wherein the modifications in the method of manufacturing are fashioned by including or excluding steps as follows: compression followed by heating, coating of the final product using hydrophobic materials or addition of a push layer followed by coating with a semi-permeable membrane.

The invention further provides a lipophilic abuse and tamper resistant drug delivery system wherein the active belongs to a group of abuse-prone opioid analgesic selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof. The invention further provides a lipophilic abuse and tamper resistant drug delivery system wherein the ion exchange resin comprises of ionizable groups attached to a polymer backbone where in the polymer backbone is usually formed by polymers, dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl or combinations thereof.

The invention further provides a lipophilic abuse and tamper resistant drug delivery system wherein the binder for granulation is lipophilic and may consist of a single or a group of lipophilic materials that belong to a group of fatty acid esters, Compritol 888 ATO, glycerylmonostereate, precirol, and combinations thereof. The invention further provides a lipophilic abuse and tamper resistant drug delivery system wherein the binder for granulation is a mixture of lipophilic substances selected from a group of fatty acid esters, Compritol 888 ATO, glycerylmonostereate, Precirol, hydrophilic substances selected from a group of water soluble or water insoluble, non-gelling binders as Poly(vinyl) pyrrolidone, Poly(vinyl) alcohol, starch, corn starch, pregelatinized starch, microcrystalline cellulose (MCC), silicified MCC, microfine cellulose, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropylmethyl celluloses, and combinations thereof.

The invention further provides a lipophilic abuse and tamper resistant drug delivery system where in the binder for granulation is a mixture of substances and is melted to achieve uniformity in blend in granulation. The invention further provides a lipophilic abuse and tamper resistant drug delivery system wherein the binders can be added intragranularly or extra granularly. The invention further provides a lipophilic abuse and tamper resistant drug delivery system where in the viscosity enhancing agents that are organic in nature, Poly(vinyl) pyrrolidone, Poly(vinyl) alcohol, viscosity enhancing agents that are inorganic in nature, Silicon dioxide, Bentonite, and combinations thereof.

The invention further provides a lipophilic abuse and tamper resistant drug delivery system wherein the lubrication agents are lipophilic and may belong to a class of fatty acids, stearic acid, mystic acid, palmitic acid, and combinations thereof. The invention further provides a lipophilic abuse and tamper resistant drug delivery system wherein the lubrication agents are lipophilic and may belong to a class of fatty acid esters that include glyceride esters, glyceryl monostearate, glyceryl tribehenate, glyceryl dibehenate, and sugar esters, sorbitan monostearate and sucrose monopalmitate. The invention further provides a lipophilic abuse and tamper resistant drug delivery system wherein the lubrication agents are metallic salts of fatty acids, magnesium, calcium or zinc salts of stearic acid, mystic acid, palmitic acid, and combinations thereof. The invention further provides a lipophilic abuse and tamper resistant drug delivery system wherein the lubrication agents belong to a class of inorganic materials, talc, calcium silicate.

The invention further provides a lipophilic abuse and tamper resistant drug delivery system wherein the tablet is coated with either hydrophilic or lipophilic coating agents, polymeric materials, derivatives of cellulose (hydroxypropylmethyl cellulose, hydroxyethyl cellulose, hydroxypropyl cellulose, and their respective derivatives), polymers of alginic acid and its salts and derivatives, derivatives of acrylic and methacrylic acid, polymers and copolymers of said acids and/or their respective esters to further retard drug extraction or to increase gastric resistance.

The invention further provides a lipophilic abuse and tamper resistant drug delivery system wherein the tablet coat presented may consist of plasticizing materials, triethyl citrate, diethyl phthalate, diacetin, triacetin, dibutyl phthalate, dibutyl tartrate, tributyl acetate, castor oil, cetyl alcohol, cetyl stearyl alcohol, fatty acids, glycerides and triglycerides and polyoxyethylene glycols. The invention further provides a lipophilic abuse and tamper resistant drug delivery system wherein the at least one active agent susceptible to abuse is present in an amount of about 10 to about 60 wt % of the composition. The invention further provides a lipophilic abuse and tamper resistant drug delivery system wherein the at least one surfactant is present in an amount of about 10 to about 60 wt % of the composition. The invention further provides a lipophilic abuse and tamper resistant drug delivery system wherein the polymer is present in an amount of about 10 to about 60 wt % of the composition.

The invention provides a product of manufacture comprising a blister package; a lidded blister; a blister card or packet; a clamshell; an intravenous (IV) package, IV packette or IV container; a tray or a shrink wrap comprising a pharmaceutical composition of the invention, and instructions for use of the pharmaceutical composition for treating and/or preventing conditions as set forth herein.

The invention provides for the use of the compositions of the invention for the production of a medicament for treating and/or preventing the indications as set forth herein.

In accordance with a further embodiment, the present invention provides a use of the pharmaceutical compositions described herein, in an amount effective for use in a medicament, and most preferably for use as a medicament for treating and/or preventing a disease or disorder in a subject.

### BRIEF DESCRIPTION OF SEVERAL VIEWS OF THE DRAWINGS

The invention will be described in conjunction with the following drawings in which like reference numerals designate like elements and wherein:

FIG. 1 shows abuse and tamper resistant osmotic drug delivery system containing therapeutic agent in ion exchange resin complex form.

FIG. 2 shows abuse and tamper resistant osmotic drug delivery system containing therapeutic agent in microencapsulated form.

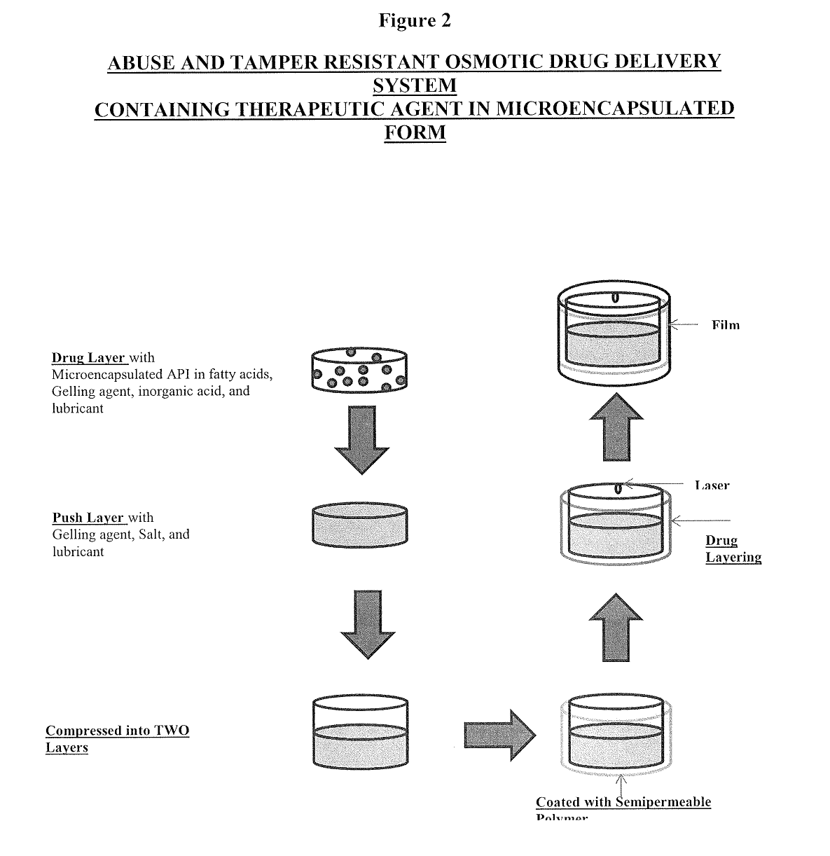

FIG. 3 is a chart showing Self-emulsified abuse and tamper resistant Oxycodone Capsules, 40 mg dissolution evaluated in pH 1.2 simulated gastric fluid (SGF) in a dissolution apparatus II (paddle) at 100 rpm and a volume of 900 ml maintained at a temperature of 37.0° C. (±0.5° C.), followed by HPLC method.

FIG. 4 is a chart showing the Comparative Release Profile of Abuse and tamper resistant immediate release solid dosage form containing Oxycodone HCl in complex form with Amberlite IRP 69. The immediate release abuse and tamper resistant tablets released drug more than 90% in 30 minutes and less than 10% in 40% ethanol dissolution media.

FIG. 5 is a depiction of the dissolution of a matrix type device.

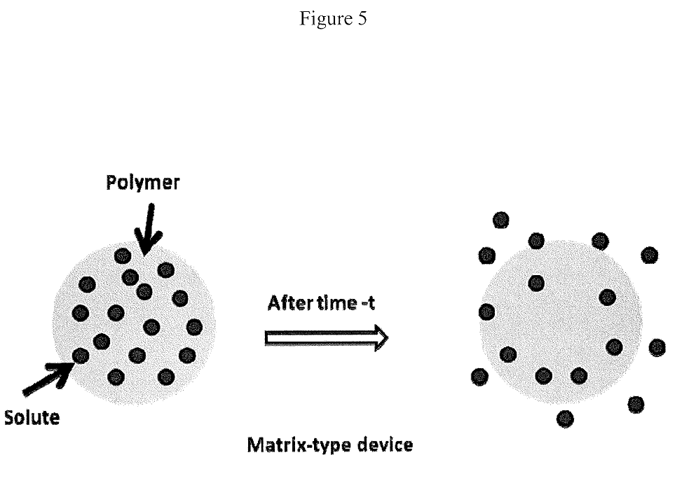

### DETAILED DESCRIPTION OF THE INVENTION

The term “gelling agent” as used herein includes a compound or composition used to impart gel-like or thickening quality to a tampered dosage form upon the addition of moisture or liquid.

“Opioids” are synthetic versions of opium. They have the ability to reduce pain but can also suppress breathing to a fatal degree when taken in excess. Examples of opioids are oxycodone (OxyContin®), oxymorphone (Opana®), and hydrocodone (Vicodin®), and methadone.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may easily be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

### Abuse and Tamper Resistant Formulations

The present invention relates to the field of abuse and tamper resistant immediate and controlled release formulations based on In-Situ Self-Emulsifying Nanosystem (i-SENS). The present invention comprises, in addition to one or more active ingredients with abuse potential (A) optionally together with physiologically acceptable one or more high and/or low HLB surfactants and co-surfactants, (B) one pH independent high and/or low viscous ethylcellulose polymer, (C) oleic acid, and (D) one or more hydrophilic solvents to formulate into the abuse and tamper resistant dosage form. The resultant highly viscous liquid can be dispensed “as is”, and/or encapsulate into a two-piece gelatin shell and/or seamless softgel capsules.

The present invention relates to the field of abuse and tamper resistant immediate and controlled release formulations comprising a drug-resin complex with one or more swellable, and non-swellable hydrophilic, non-swellable lipophilic and wax polymeric matrix followed by curing between 60° C. and 85° C. for 5 seconds to 300 seconds.

The present invention comprises, in addition to one or more active ingredients with abuse potential (A) optionally together with physiologically acceptable one or more high and/or low molecular weight water soluble polyethylene swellable polymers, (B) one or more non-swellable low molecular polyethylene glycols, and one or more waxes to formulate into an abuse and tamper resistant dosage form.

The kinetic drug release from matrix tablets follows the Fickian law of diffusion, which is driven by the particle size of resinate, amount of polymer, degree of swelling and concentration gradient exist between tablet and surrounding media. Furthermore, according to this invention, the kinetic drug release from the tablet is modulated by curing process between 60° C. and 85° C. for 5 seconds to 300 seconds.

In other embodiments, the present invention relates to abuse and tamper resistant controlled release solid dosage forms containing at least one therapeutic agent susceptible to abuse in an ion exchange resin complex form. Furthermore, the present invention contains, for example, low molecular hydrophilic polyethylene oxide, a water soluble ionic compound, and non-digestible wax.

The present invention further discloses an abuse and tamper resistant lipophilic drug delivery system predominantly administered orally and encompassing at least one opioid active prone to substance abuse by alcoholic extraction and/or other tampering methods including crushing, and grinding. Furthermore, the invention involves phenomenon most commonly referred to as “Sintering”, wherein an active agent adheres to an adjacent lipophilic surface in a mass of powder and/or in a compact by heat and/or compression and/or compression followed by heat. The said abuse and tamper resistant dosage form comprises one or more functional excipients in an effective amount to make the dosage form unsuitable for administration of parenteral and nasal administration. The preparation involves granulation of an active agent susceptible to abuse, such as an opioid, using a mixture of hydrophilic and lipophilic binders in a solid or liquid state at regular or elevated temperatures to form granules that modulate the release of the active for abuse intended extraction along with lubrication and coating agents.

### Active Pharmaceutical Agents

The formulations of the invention are particularly well-suited for oral dosage units containing drugs having abuse potential, such as opioids. In one embodiment, these oral dosage units are solid dosage units. However, these formulations are adaptable to other types of dosage units (e.g., suspensions, etc.) and other active components.

In one embodiment, the drugs susceptible to abuse such as opioids are used in the treatment of respiratory tract disorders such as, for example, antitussive expectorants such as dihydrocodeine phosphate, codeine phosphate, and noscapine hydrochloride. In another embodiment, the opioid drugs are analgesics drugs such as hydrocodone, morphine, hydromorphone, oxycodone, codeine, levorphanol, meperidine, methadone, oxymorphone, buprenorphine, fentanyl and derivatives thereof, dipipanone, tramadol, etorphine, dihydroetorphine, butorphanol, levorphanol, or salts thereof or mixtures thereof. In one embodiment, the opioid is morphine, oxycodone, hydrocodone, or a salt thereof. In one embodiment, a morphine salt is morphine sulfate; an oxycodone salt is oxycodone HCl; and a codeine salt is codeine sulfate or phosphate.

In certain embodiments, the amount of the active drugs susceptible to abuse in the composition may be about 1 mg to 250 mg. In another embodiment, the amount of the active drug in the composition is about 5 mg to about 200 mg. In still another embodiment, the amount of the drug drugs susceptible to abuse in the composition is about 10 mg to about 150 mg. The preceding list is not intended to be exclusive. In some embodiments, the composition of the invention is designed to provide a release profile similar to a commercially available product. In such an instance, the present invention provides an equivalent amount of active to the commercially available product based on weight. In another embodiment, the present invention provides an amount of active bioequivalent to the commercially available product, i.e., provides a finished formulation having an in vivo release profile similar that of the commercial product. This can be readily determined by taking into consideration the molecular weight of the free base of the active drug hound to the resin, as compared to the compound in the commercial product, and further taking into consideration the percentage of active drug loaded on the resin. These calculations are well within the skill of one in the art.

Examples of specific active drug substances suitable for use in the pharmaceutical compositions provided herein include: anti-inflammatory and antirheumatic active drug substances, such as, for example: butylpyrazolidine, phenylbutazone, mofebutazone, oxyphenbutazone, clofezone, kebuzone, acetic acid derivatives and related substances, indometacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac, bufexamac, oxicam, piroxicam, tenoxicam, droxicam, lornoxicam, meloxicam, methotrexate, propionic acid derivatives, ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, fenamates, mefenamic acid, tolfenamic acid, flufenamic acid, meclofenamic acid, coxibs, celecoxib, rofecoxib, valdecoxib, parecoxib, etoricoxib, lumiracoxib, nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, feprazone, dipyrocetyl, acetylsalicylic acid, quinolines, oxycinchophen, gold preparations, sodium aurothiomalate, sodium aurotiosulfate, auranofin, aurothioglucose, aurotioprol, penicillamine or bucillamine.

In another embodiment, suitable active pharmaceutical ingredients can comprise analgesics, such as, for example: opioids, natural opium alkaloids, morphine, opium, hydromorphone, nicomorphine, oxycodone, dihydrocodeine, diamorphine, tapentadol, papavereturn, codeine, phenylpiperidine derivatives, ketobemidone, pethidine, fentanyl, diphenylpropylamine derivatives, dextromoramide, piritramide, dextropropoxyphene, bezitramide, methadone, benzomorphan derivatives, pentazocine, phenazocine, oripavine derivatives, buprenorphine, morphinan derivatives, butorphanol, nalbuphine, tilidine, tramadol, dezocine, salicylic acid and derivatives, acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, pyrazolones, phenazone, metamizole sodium, aminophenazone, propyphenazone, nifenazone, anilides, paracetamol, phenacetin, bucetin, propacetamol, other analgesics and antipyretics, such as, for example: rimazolium, glafenine, floctafenine, viminol, nefopam, flupirtine, or ziconotide.

In another embodiment, suitable active pharmaceutical ingredients can comprise anaesthetics, such as, for example: ethers, diethyl ether, vinyl ether, halogenated hydrocarbons, halothane, chloroform, methoxyflurane, enflurane, trichloroethylene, isoflurane, desflurane, sevoflurane, barbiturates, methohexital, hexobarbital, thiopental, narcobarbital, opioid anaesthetics, fentanyl, alfentanil, sufentanil, phenoperidine, anileridine, remifentanil, other general anaesthetics, such as, for example: droperidol, ketamine, propanidid, alfaxalone, etomidate, propofol, hydroxybutyric acid, nitrous oxide, esketamine, xenon, esters of aminobenzoic acid, metabutethamine, procaine, tetracaine, chloroprocaine, benzocaine, amides, bupivacaine, lidocaine, mepivacaine, prilocaine, butanilicaine, cinchocaine, etidocaine, articaine, ropivacaine, levobupivacaine, esters of benzoic acid, cocaine, other local anaesthetics, such as, for example: ethyl chloride, dyclonine, phenol, or capsaicin.

In another embodiment, suitable active pharmaceutical ingredients can comprise antimigraine active drug substances, such as, for example: ergot alkaloids, dihydroergotamine, ergotamine, methysergide, lisuride, corticosteroid derivatives, flumedroxone, selective serotonin (5HT.sup.1) agonists, sumatriptan, naratriptan, zolmitriptan, rizatriptan, almotriptan, eletriptan, frovatriptan, other antimigraine preparations, pizotifen, clonidine, iprazochrome, dimetotiazine, or oxetorone.

In another embodiment, suitable active pharmaceutical ingredients can comprise antiepileptic active drug substances, such as, for example: barbiturates and derivatives, methylphenobarbital, phenobarbital, primidone, barbexaclone, metharbital, hydantoin derivatives, ethotoin, phenytoin, amino(diphenylhydantoin) valeric acid, mephenytoin, fosphenytoin, oxazolidine derivatives, paramethadione, trimethadione, ethadione, succinimide derivatives, ethosuximide, phensuximide, mesuximide, benzodiazepine derivatives, clonazepam, carboxamide derivatives, carbamazepine, oxcarbazepine, rufinamide, fatty acid derivatives, valproic acid, valpromide, aminobutyric acid, vigabatrin, progabide, tiagabine, other antiepileptics, such as, for example: sultiame, phenacemide, lamotrigine, felbamate, topiramate, gabapentin, pheneturide, levetiracetam, zonisamide, pregabalin, stiripentol, lacosamide, or beclamide.

In another embodiment, suitable active pharmaceutical ingredients can comprise anticholinergic active drug substances, such as, for example: tertiary amines, trihexyphenidyl, biperiden, metixene, procyclidine, profenamine, dexetimide, phenglutarimide, mazaticol, bornaprine, tropatepine, ethers chemically close to antihistamines, etanautine, orphenadrine (chloride), ethers of tropine or tropine derivatives, benzatropine, or etybenzatropine.

In another embodiment, suitable active pharmaceutical ingredients can comprise dopaminergic active drug substances, such as, for example: dopa and dopa derivatives, levodopa, melevodopa, etilevodopa, adamantine derivatives, amantadine, dopamine agonists, bromocriptine, pergolide, dihydroergocryptine mesylate, ropinirole, pramipexole, cabergoline, apomorphine, piribedil, rotigotine, monoamine, oxidase B inhibitors, selegiline, rasagiline, other dopaminergic agents, such as, for example: tolcapone, entacapone, or budipine.

In another embodiment, suitable active pharmaceutical ingredients can comprise antipsychotic active drug substances, such as, for example: phenothiazines with aliphatic side-chain, chlorpromazine, levomepromazine, promazine, acepromazine, triflupromazine, cyamemazine, chlorproethazine, phenothiazines with piperazine structure, dixyrazine, fluphenazine, perphenazine, prochlorperazine, thiopropazate, trifluoperazine, acetophenazine, thioproperazine, butaperazine, perazine, phenothiazines with piperidine structure, periciazine, thioridazine, mesoridazine, pipotiazine, butyrophenone derivatives, haloperidol, trifluperidol, melperone, moperone, pipamperone, bromperidol, benperidol, droperidol, fluanisone, indole derivatives, oxypertine, molindone, sertindole, ziprasidone, thioxanthene derivatives, flupentixol, clopenthixol, chlorprothixene, tiotixene, zuclopenthixol, diphenylbutylpiperidine derivatives, fluspirilene, pimozide, penfluridol, diazepines, oxazepines, thiazepines, loxapine, clozapine, olanzapine, quetiapine, neuroleptics, tetrabenazine, benzamides, sulpiride, sultopride, tiapride, remoxipride, amisulpride, veralipride, levosulpiride, lithium, other antipsychotics, such as, for example prothipendyl, risperidone, clotiapine, mosapramine, zotepine, aripiprazole, or paliperidone.

In another embodiment, suitable active pharmaceutical ingredients can comprise anxiolytic active drug substances, such as, for example: benzodiazepine derivatives, diazepam, chlordiazepoxide, medazepam, oxazepam, potassium clorazepate, lorazepam, adinazolam, bromazepam, clobazam, ketazolam, prazepam, alprazolam, halazepam, pinazepam, camazepam, nordazepam, fludiazepam, ethyl loflazepate, etizolam, clotiazepam, cloxazolam, tofisopam, diphenylmethane derivatives, hydroxyzine, captodiame, carbamates, meprobamate, emylcamate, mebutamate, dibenzo-bicyclo-octadiene derivatives, benzoctamine, azaspirodecanedione derivatives, buspirone, other anxiolytics, such as, for example: mephenoxalone, gedocarnil, or etifoxine.

In another embodiment, suitable active pharmaceutical ingredients can comprise hypnotic and sedative active drug substances, such as, for example: barbiturates, pentobarbital, amobarbital, butobarbital, barbital, aprobarbital, secobarbital, talbutal, vinylbital, vinbarbital, cyclobarbital, heptabarbital, reposal, methohexital, hexobarbital, thiopental, ethallobarbital, allobarbital, proxibarbal, aldehydes and derivatives, chloral hydrate, chloralodol, acetylglycinamide chloral hydrate, dichloralphenazone, paraldehyde, benzodiazepine emepronium derivatives, flurazepam, nitrazepam, flunitrazepam, estazolam, triazolam, lormetazepam, temazepam, midazolam, brotizolam, quazepam, loprazolam, doxefazepam, cinolazepam, piperidinedione derivatives, glutethimide, methyprylon, pyrithyldione, benzodiazepine related drugs, zopiclone, zolpidem, zaleplon, ramelteon, other hypnotics and sedatives, such as, for example: methaqualone, clomethiazole, bromisoval, carbromal, scopolamine, propiomazine, triclofos, ethchlorvynol, valerian, hexapropymate, bromides, apronal, valnoctamide, methylpentynol, niaprazine, melatonin, dexmedetomidine, or dipiperonylaminoethanol.

In another embodiment, suitable active pharmaceutical ingredients can comprise antidepressant active drug substances, such as, for example: non-selective monoamine reuptake inhibitors, desipramine, imipramine, imipramine oxide, clomipramine, opipramol, trimipramine, lofepramine, dibenzepin, amitriptyline, nortriptyline, protriptyline, doxepin, iprindole, melitracen, butriptyline, dosulepin, amoxapine, dimetacrine, amineptine, maprotiline, quinupramine, selective serotonin reuptake inhibitors, zimeldine, fluoxetine, citalopram, paroxetine, sertraline, alaproclate, fluvoxamine, etoperidone, escitalopram, monoamine oxidase inhibitors, isocarboxazid, nialamide, phenelzine, tranylcypromine, iproniazide, iproclozide, monoamine oxidase A inhibitors, moclobemide, toloxatone, other antidepressants, such as, for example: oxitriptan, tryptophan, mianserin, nomifensine, trazodone, nefazodone, minaprine, bifemelane, viloxazine, oxaflozane, mirtazapine, medifoxamine, tianeptine, pivagabine, venlafaxine, milnacipran, reboxetine, gepirone, duloxetine, agomelatine, desvenlafaxine, centrally acting sympathomimetics, such as, for example: amfetamine, dexamfetamine, lisdexamfetamine, metamfetamine, methylphenidate, dexmethylphenidate, pemoline, fencamfamin, modafinil, fenozolone, atomoxetine, fenetylline, xanthine derivatives, caffeine, propentofylline, other psychostimulants and nootropics, such as, for example meclofenoxate, pyritinol, piracetam, deanol, fipexide, citicoline, oxiracetam, pirisudanol, linopirdine, nizofenone, aniracetam, acetylcarnitine, idebenone, prolintane, pipradrol, pramiracetam, adrafinil, or vinpocetine.

In another embodiment, suitable active pharmaceutical ingredients can comprise anti-dementia active drug substances, such as, for example: anticholinesterases, tacrine, donepezil, rivastigmine, galantamine, other anti-dementia drugs, memantine, or ginkgo biloba.

In another embodiment, suitable active pharmaceutical ingredients can comprise other nervous system active drug substances, such as, for example: parasympathomimetics, anticholinesterases, neostigmine, pyridostigmine, distigmine, ambenonium, choline esters, carbachol, bethanechol, and other parasympathomimetics, such as, for example, pilocarpine, or choline alfoscerate.

Active drug substances used in addictive disorders, such as, for example: nicotine, bupropion, varenicline, disulfiram, calcium carbimide, acamprosate, naltrexone, buprenorphine, methadone, levacetylmethadol, lofexidine, betahistine, cinnarizine, flunarizine, acetylleucine, gangliosides and ganglioside derivatives, tirilazad, riluzole, xaliproden, hydroxybutyric acid, or amifampridine.

In another embodiment, suitable active pharmaceutical ingredients can comprise opium alkaloids and derivatives, such as, for example: ethylmorphine, hydrocodone, codeine, opium alkaloids with morphine, normethadone, noscapine, pholcodine, dextromethorphan, thebacon, dimemorfan, acetyldihydrocodeine, benzonatate, benproperine, clobutinol, isoaminile, pentoxyverine, oxolamine, oxeladin, clofedanol, pipazetate, bibenzonium bromide, butamirate, fedrilate, zipeprol, dibunate, droxypropine, prenoxdiazine, dropropizine, cloperastine, meprotixol, piperidione, tipepidine, morclofone, nepinalone, levodropropizine, or dimethoxanate.

In another embodiment, the active pharmaceutical ingredient may be a substance with abuse potential that presents a safety risk. Such active drug substance may include: 1-(1-phenylcyclohexyl)pyrrolidine, 1-(2-phenylethyl)-4-phenyl-4-acetoxypiperidine, 1-[1-(2-thienyl)-cyclohexylpiperidine, 1-[1-(2-thienyl)cyclohexyl]pyrrolidine, 1-methyl-4-phenyl-4-propionoxy-piperidine, 1-phenylcyclohexylamine, 1-piperidinocyclohexanecarbonitrile, 2,5-dimethoxy-4-ethylamphetamine, 2,5-dimethoxyamphetamine, 2C-B-(4-bromo-2,5-dimethoxyphenethylamine), 2C-D (2,5-dimethoxy-4-methylphenethylamine), 2C-I (4-iodo-2,5-dimethoxy-phenethylamine), 2C-T-2 (2,5-dimethoxy-4-ethylthiophenethylamine), 2C-T-4 (2,5-dimethoxy-4-isopropyl thiophenethylamine), 2C-T-7 (2,5-dimethoxy-4-(n)-propylthiophenethylamine), 3,4-methylene-dioxymethamphetamine, 3,4,5-trimethoxyamphetamine, 3,4-methylenedioxyamphetamine, 3,4-methylenedioxy-N-ethyl amphetamine, 3-methylfentanyl, 3-methylthiofentanyl, 4-bromo-2,5-dimethoxyamphetamine, 4-bromo-2,5-dimethoxyphenethylamine, 4-methoxyamphetamine, 4-methyl-2,5-dimethoxyamphetamine, 4-methylaminorex (cis isomer), 5-MeO-DIPT (5-methoxy-N,N-diisopropyltryptamine), 5-MeO-DMT (5-methoxy-N,N-dimethyltryptamine), 5-methoxy-3,4-methylenedioxyamphetamine, acetorphine, acetorphine, acetyl-alpha-methylfentanyl, acetyl-alpha-methylfentanyl, acetyldihydrocodeine, acetylmethadol, acetylmethadol, alfentanil, allobarbital, allylprodine, alphacetylmethadol except levo-alphacetylmethadol, alpha-ethyltryptamine, alphameprodine, alphamethadol, alphamethadol, alpha-methylfentanyl, alpha-methylthiofentanyl, alphaprodine, alprazolam, amfepramon, amfetaminil, amineptin, aminorex, amobarbital, amphetamine, dextroamphetamine, amilnitrite (all isomers of the amyl group), anabolic steroids, anileridine, aprobarbital, barbital, barbituric acid derivative, BDB (3,4-methylenedioxyphenyl)-2-butanamine), benzethidin, benzethidine, benzoylecgonine, benzphetamine, benzphetamine, benzylmethylcetone, benzylmorphine, betacetylmethadol, beta-hydroxy-3-methylfentanyl, beta-hydroxyfentanyl, betameprodine, betameprodine, betamethadol, betaprodine, bezitramide, bezitramide, boldenone, brolamfetamine, bromazepam, brotizolam, bufotenine, buprenorphine, butabarbital, butalbital, butobarbital, butorphanol, BZP (A2)(1-benzylpiperazin), camazepam, cannabis, carfentanil, catha edulis, cathine, cathinone, chloral betaine, chloral hydrate, chlordiazepoxide, chlorhexadol, chlorotestosterone (same as clostebol), chlorphentermine, clobazam, clonazepam, clonitazene, clonitazene, clorazepate, clortermine, clostebol, clotiazepam, cloxazolam, coca leaves, cocaine, codeine, codeine and isoquinoline alkaloid, codeine methylbromide, codeine-N-oxide, codoxime, cyclobarbital (hexemal NFN), cyprenorphine, dehydrochlormethyltestosterone, delorazepam, desomorphine, dexamfetamine, dexfenfluramine, dexmethylphenidate, dextromoramide, dextropropoxyphene, diacetylmorphine, diampromide, diazepam, dichloralphenazone, diethylpropion, diethylthiambutene, diethyltryptamine, difenoxin, dihydrocodeine, dihydroetorphine, dihydromorphine, dihydrotestosterone, dimenoxadol, dimepheptanol, dimethylthiambutene, dimethyltryptamine, dioxaphetyl butyrate, diphenoxylate, dipipanone, diprenorphine, dronabinol, drostanolone, drotebanol, ecgonine, estazolam, ethchlorvynol, ethinamate, ethyl loflazepate, ethylestrenol, ethylmethylthiambutene, ethylmorphine, ethylmorphine, eticyclidine, etilamfetamine, etonitazene, etorphine, etoxeridine, etryptamine, fencamfamin, fenethylline, fenetylline, fenfluramine, fenproporex, fentanyl, fludiazepam, flunitrazepam, fluoxymesterone, flurazepam, formebolone, fungi and spores of the species psilocybe semilanceata, furethidine, gamma hydroxybutyric acid, glutethimide, halazepam, haloxazolam, heroine, hydrocodone, hydrocodone & isoquinoline alkaloid, hydromorphinol, hydromorphone, hydroxypethidine, ibogaine, isobutyl nitrite, isomethadone, ketamine, ketazolam, ketobemidone, levamfetamine, levo-alphacetylmethadol, levo-methamphetamine, levomethorphan, levomoramide, levophenacylmorphan, levorphanol, lisdexamfetamine, loprazolam, lorazepam, lormetazepam, lysergic acid, lysergic acid amide, lysergic acid diethylamide, marijuana, mazindol, MBDN (N-methyl-1-(3,4-methylenedioxyphenyl)-2-butanamine), mCPP (1-(3-chlorphenyl)piperazine), mebutamate, mecloqualone, medazepam, mefenorex, MeOPP (1-(4-methoxyphenyl)piperazine), meperidine, meperidine intermediate, meprobamate, mescaline, mesocarb, mesterolone, metamfetamine, metazocine, methadone, methadone intermediate, methamphetamine, methandienone, methandrolone, methandriol, methandrostenolone, methaqualone, methcathinone, methenolone, methohexital, methyldesorphine, methyldihydromorphine, methylphenidate, methylphenobarbital (mephobarbital), methyltestosterone, methyprylone, metopone, mibolerone, midazolam, modafinil, moramide-intermediate, morpheridine, morphine, morphine methylbromide, morphine methylsulfonate, morphine-N-oxide, myrophine, N,N-dimethylamphetamine, nabilone, nalorphine, nandrolone, N-ethyl-1-phenylcyclohexylamine, N-ethyl-3-piperidyl benzilate, N-ethylamphetamine, N-hydroxy-3,4-methylenedioxyamphetamine, nicocodeine, nicocodine, nicodicodine, nicomorphine, nimetazepam, nitrazepam, N-methyl-3-piperidyl benzilate, noracymethadol, norcodeine, nordiazepam, norethandrolone, norlevorphanol, normethadone, normorphine, norpipanone, norpipanone, opium, oxandrolone, oxazepam, oxazolam, oxycodone, oxymesterone, oxymetholone, oxymorphone, para-fluorofentanyl, parahexyl, paraldehyde, pemoline, pentazocine, pentobarbital, petrichioral, peyote, phenadoxone, phenampromide, phenazocine, phencyclidine, phendimetrazine, phenmetrazine, phenobarbital, phenomorphan, phenoperidine, phentermine, phenylacetone, pholcodine, piminodine, pinazepam, pipradrole, piritramide, PMMA (paramethyxymethyl amphetamine), prazepam, proheptazine, properidine, propiram, psilocybine, psilocine, pyrovalerone, quazepam, racemethorphane, racemoramide, racemorphane, remifentanil, salvia divinorum, salvinorin A, secobarbital, secobarbital, sibutramine, SPA, stanolone, stanozolol, sufentanil, sulfondiethylmethane, sulfonethylmethane, sulfonmethane, talbutal, temazepam, tenamfetamine, testolactone, testosterone, tetrahydrocannabinols, tetrazepam, TFMPP (1-(3-triflourmethylphenyl)piperazine), thebacon, thebaine, thiamylal, thiofentanyl, thiopental, tiletamine and zolazepam in combination, tilidine, trenbolone, triazolam, trimeperidine, vinbarbital, zaleplon, zipeprol, zolpidem, or zopiclone.

Other suitable examples of active drug substances suitable for use in the pharmaceutical compositions described herein include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, dextropropoxyphene, ketobemidone, levallorphan, levorphanol, levophenacylmorphan, lofentanil, meperidine, meptazinol, metazocine, methadone, metopon, morphine, morphine 6-glucuronide, morphine 3-glucuronide, myrophine, nalbuphine, narcine, nicomorphine, norlevorphanol, normethadone, nalorphine, normorphine, norpipanone, opium, oxycodone, oxycodeine, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, tramadol, thebaine, levo-alphacetylmethadol (LAAM), remifentanil, carfentanyl, ohmefentanyl, MPPP, prodine, PEPAP, levomethorphan, etorphine, lefetamine, loperamide, diphenoxylate, or pethidine.

Other examples of active drug substances suitable for use in the pharmaceutical compositions described herein include anabolic steroids, cannabis, cocaine, or diazepam.

In another embodiment, the active drug substance comprises the therapeutic classes including non-steroidal anti-inflammatory substances or antirheumatic active drug substances.

In other embodiments, the active drug substance comprises analgesics, opioids, antipyretics, anaesthetics, antimigraine agents, antiepileptics, anti-parkinson agents, dopaminergic agents, antipsychotics, anxiolytics, sedatives, antidepressants, psychostimulants agents, dopamine, noradrenaline, nicotinic, alfa-adrenergic, serotonin, H3 antagonists used for ADHD or nootropics agents used in addictive disorders.

In other embodiments, the active drug substance comprises therapeutic classes including anaesthetics, centrally acting analgesics, sedative-hypnotics, anxiolytics, appetite suppressants, decongestants, antitussives, antihistamines, antiemetics, antidiarrheals, and drugs used to treat narcolepsy, or attention deficit hyperactivity disorder.

In another embodiment, the active drug substance is associated with abuse syndromes and the active drug substance may, for example, be selected from opioids, CNS depressants, CNS stimulants, cannabinoids, nicotine-like compounds, glutamate antagonists, or N-methyl-D-aspartate (NMDA) antagonists.

In another embodiment, the active drug substance is an analgesic. Examples of analgesics suitable for use in the pharmaceutical compositions described herein include, for example, opioids, natural opium alkaloids, morphine, opium, hydromorphone, nicomorphine, oxycodone, dihydrocodeine, diamorphine, tapentadol, papavereturn, codeine, phenylpiperidine derivatives, ketobemidone, pethidine, fentanyl, diphenylpropylamine derivatives, dextromoramide, piritramide, dextropropoxyphene, bezitramide, methadone, benzomorphan derivatives, pentazocine, phenazocine, oripavine derivatives, buprenorphine, morphinan derivatives, butorphanol, nalbuphine, tilidine, tramadol, dezocine, salicylic acid and derivatives, acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, pyrazolones, phenazone, metamizole sodium, aminophenazone, propyphenazone, nifenazone, anilides, paracetamol, phenacetin, bucetin, propacetamol, other analgesics and antipyretics such as, for example, rimazolium, glafenine, floctafenine, viminol, nefopam, flupirtine, or ziconotide.

In another embodiment, the active drug substance is an opioid. Where an opioid is included as an active drug substance, the opioid may comprise naturally occurring opioids, synthetic opioids, or semisynthetic opioids.

In other embodiment, the active drug substance comprises amfetamine, dexamfetamine, lisdexamfetamine, metamfetamine, methylphenidate, dexmethylphenidate, or combinations thereof.

Where an opioid is used as an active drug substance, the opioid may be present in any of its crystalline, polymorphous, semi-crystalline, and amorphous or polyamorphous forms. Furthermore, in another embodiment, an opioid used as an active drug substance may be present in one or more forms selected from its crystalline, polymorphous, semi-crystalline, or amorphous or polyamorphous forms.

Pharmaceutically Acceptable Salt

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. The pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like. Other suitable salts will be readily apparent to one of skill in the art. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine.

### Ion Exchange Resin

Ion exchange resins are made up of a polymeric structure acting as a support for acidic or basic polar functional groups, including ions that can be exchanged with other ions present in the medium. Said resins are classified as cation or anion exchange resins, depending on the type of ions exchanged: cations (Na⁺ or Ca²⁺, for example) or anions (Cl⁻ or OH⁻, for example), respectively.

Ion-exchange resins suitable for use in the preparations of the invention may be water-insoluble and comprise a preferably pharmacologically inert organic and/or inorganic matrix containing functional groups that are ionic or capable of being ionized under the appropriate conditions of pH. Typically the size of the ion-exchange particles is from about 10 microns to about 420 microns, preferably the particle size is within the range of about 40 microns to about 250 microns for solid dosage forms, e.g., tablets and granules placed in capsules. Particle sizes substantially below the lower limit are generally difficult to handle in all steps of the processing. Generally, uncoated drug-ion exchange resin particles of the invention will tend to be at the lower end of this range, whereas coated drug-ion exchange resin particles of the invention will tend to be at the higher end of this range. However, both uncoated and coated drug-ion exchange resin particles may be designed within this size range.

A suitable ion exchange resin is selected depending upon the charge of the active agent or its salt. For example, cation exchange resins are well suited for use with drugs and other molecules having a cationic functionality, including, e.g., oxycodone, morphine, hydrocodone, oxymorphone, and hydromorphone, as well as prodrugs, salts, isomers, polymorphs, and solvates thereof. Cationic exchange resins have been described in the art and also commercially available. Examples of commercially available cationic resins include, without limitation, Dow XYS-40010.00 and Dow XYS-40013.00 (The Dow Chemical Company), Amberlite IRP-69 (an insoluble, strongly acidic, sodium polystyrene cation exchange resin), Amberlite IRP-64 (weekly acidic), Amberlite IRP-120 (Rohm and Haas), Amberlite IRP-88 (weakly acidic). Amberlite IRP-69 (Rohm and Haas) is sulfonated polymers composed of polystyrene crosslinked with 8% of divinylbenzene, with an ion exchange capacity of about 4.5 to 5.5 meq/g of dry resin. It consists of irregularly shaped particles with a size range of 47 to 149 microns. A series of cationic resins are also available from DOW Chemical as the DOWEX™ 50WX series (Dow Chemical Company). There are mainly four products with different particle size distribution: cut-off mesh size is US Sieve No. 50 (300 microns) in the case of Dowex™ 50WX2-50, 100 (150 microns) in Dowex™ 50WX2-100, 200 (75 microns) in Dowex™ 50 WX2-200, and 400 (38 microns) in Dowex™ 50WX2-400. Crosslinking is another important factor, which can influence physical properties, equilibrium conditions, drug loading, and drug release profiles. Resins of various degrees of permeability are dependent on the divinylbenzene content, which was described as the degree of resin crosslinkage and the number after X is the percentage of divinylbenzene in the resin polymer. For example, Dowex™ 50WX2-50 contains 2% divinylbenzene with particle size is bigger than 50 mesh. Total exchange capacity of 2, 4 and 8% crosslinkage resins are 0.6, 1.1 and 1.7 meq/ml, respectively. Still other ion exchange resins are available from Sigma-Aldrich.

Both strongly acidic and weakly acidic resins (e.g., cationic resins) are commercially available and can be selected for use. However, it will be understood that the strength of the bond between the drug and the resin will be affected by whether the resin is strongly acidic or weakly acidic. More particularly, a stronger bond will typically be formed by the strongly acidic resin and thus, drugs loaded thereon will have a slower release profile than those loaded on a weakly acidic resin. Thus, one of skill in the art can select the desired type of resin to achieve a desired release profile, further taking into consideration such factors as the use of a release retardant, the thickness of the hybrid coating, and the ratio of barrier coating component to enteric coating component.

Other suitable resins can be selected by one of skill in the art, taking into consideration the charge of the free base or salt form of a desired opioid drug.

In the composition of the invention, the ion exchange resin for therapeutic use may be selected from the group consisting of sodium polystyrene sulfonate, calcium polystyrene sulfonate, cholestyramine, colesevelam, colestipol, and sevelamer. In another aspect, the ion-exchange mechanism is controlled by at least one ion-exchange resin. In another aspect, said at least one ion-exchange resin is selected from Cholestyramine, Colestipol, Sodium polystyrene sulfonate, Polacrilex resin, and/or Polacrilin potassium.

### Drug-Ion Exchange Resin Complexes

Binding of the selected drug or a combination of drugs to the ion exchange resin can be accomplished using methods known in the art.

The amount of drug that can be loaded onto a resin will typically range from about 1% to about 75% by weight of the drug-ion exchange resin particles. A skilled artisan with limited experimentation can determine the optimum loading for any drug resin complex, taking into such consideration as the desired amount of active drug and the desired size of the final dose formulation. For example, to reduce the size of a formulation or to increase the amount of active drug, a higher loading percentage may be used. Conversely, where a lesser amount of active drug is desired, a loading-percentage at the lower end of this range may be provided. In one embodiment, loading of about 10% to about 40% by weight, more desirably, about 15% to about 30% by weight, of the drug-ion exchange resin particles can be employed. Typical loadings of about 25% by weight of the drug-ion exchange resin particles can be advantageously employed.

Thus, in one aspect, the invention provides drug-ion exchange resin complexes comprising a drug loaded in an ion exchange resin as described herein. The drugs and ion exchange resins may be readily selected from amongst those drugs and resins described herein.

### Gelling Agents

In certain embodiments of the present invention the dosage form includes a gelling agent, various gelling agents can be employed including, for example and without limitation, sugars or sugar derived alcohols, such as mannitol, sorbitol, and the like, starch and starch derivatives, cellulose derivatives, such as microcrystalline cellulose, sodium caboxymethyl cellulose, methylcellulose, ethyl cellulose, hydroxyethyl cellulose, hydroxypropyl cellulose, and hydroxypropyl methylcellulose, attapulgites, bentonites, dextrins, alginates, carrageenan, gum tragacanth, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof, etc. In certain preferred embodiments, the gelling agent is xanthan gum. In other preferred embodiments, the gelling agent of the present invention is pectin. The pectin or pectic substances useful for this invention include not only purified or isolated pectates but also crude natural pectin sources, such as apple, citrus or sugar beet residues which have been subjected, when necessary, to esterification or de-esterification, e.g., by alkali or enzymes. Preferably, the pectins used in this invention are derived from citrus fruits such as lime, lemon, grapefruit, and orange.

With the inclusion of a gelling agent in the dosage form, when the dosage form is tampered with, the gelling agent preferably imparts a gel-like quality to the tampered dosage form which preferably spoils or hinders the pleasure of obtaining a rapid high from the tampered dosage form due to the gel like consistency in contact with the mucous membrane, and in certain embodiments, prevents the abuse of the dosage form by minimizing absorption, e.g. in the nasal passages. A gelling agent may be added to the formulation in a ratio of gelling agent to opioid agonist of from about 1:40 to about 40:1 by weight, preferably from about 1:1 to about 30:1 by weight, and more preferably from about 2:1 to about 10:1 by weight of the opioid agonist. In certain alternative embodiments, the gelling agent may be present in a ratio to the opioid agonist of from about 1:15 to about 15:1, preferably in a ratio of from about 1:8 to about 8:1, and more preferably from about 1:3 to about 3:1 by weight of the opioid agonist.

In certain other embodiments, the dosage form forms a viscous gel after the dosage form is tampered with, dissolved in an aqueous liquid (from about 0.5 to about 10 ml and preferably from 1 to about 5 ml), causing the resulting mixture to have a viscosity of at least about 20 cP. Most preferably, the resulting mixture will have a viscosity of at least about 50 cP, about 60 cP, about 70 cP, about 80 cP, about 90 cP, or about 100 cP.

In certain other embodiments, the dosage form forms a viscous gel after the dosage form is tampered with, dissolved in an aqueous liquid (from about 0.5 to about 10 ml and preferably from 1 to about 5 ml) and then heated (e.g., greater than about 45° C.), causing the resulting mixture to have a viscosity of at least about 20 cP. Most preferably, the resulting mixture will have a viscosity of at least about 50 cP, about 60 cP, about 70 cP, about 80 cP, about 90 cP, or about 100 cP.

### Surfactants

The composition of the invention may include a surfactant. The surfactants for use in connection with the present invention include, but are not limited to, sorbitan fatty acid esters (e.g., Spans®), polyoxyethylene sorbitan fatty acid esters (e.g., Tweens®), sodium lauryl sulfate (SLS), sodium dodecylbenzene sulfonate (SDBS) dioctyl sodium sulfosuccinate (Docusate), dioxycholic acid sodium salt (DOSS), Sorbitan Monostearate, Sorbitan Tristearate, hexadecyltrimethyl ammonium bromide (HTAB), Sodium N-lauroylsarcosine, Sodium Oleate, Sodium Myristate, Sodium Stearate, Sodium Palmitate, Gelucire 44/14, ethylenediamine tetraacetic acid (EDTA), Vitamin E d-alpha tocopheryl polyethylene glycol 1000 succinate (TPGS), Lecithin, M W 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, Pluronic F68, Pluronic F108, and Pluronic F127 (or any other polyoxyethylene-polyoxypropylene co-polymers (Pluronics®) or saturated polyglycolized glycerides (Gelucirs®)). Specific example of such surfactants that may be used in connection with this invention include, but are not limited to, Span 65, Span 25, Tween 20, Capryol 90, Pluronic F108, sodium lauryl sulfate (SLS), Vitamin E TPGS, pluronics and copolymers. SLS is generally preferred.

The amount of the surfactant relative to the total weight of the composition may be between 0.1-15%. Preferably, it is from about 0.5% to about 10%, more preferably from about 0.5 to about 5%, e.g., about 0.5 to 4%, about 0.5 to 3%, about 0.5 to 2%, about 0.5 to 1%, or about 0.5%.

In certain embodiments, the amount of the surfactant relative to the total weight of the composition is at least about 0.1%, preferably about 0.5%. In these embodiments, the surfactant would be present in an amount of no more than about 15%, and preferably no more than about 12%, about 11%, about 10%, about 9%, about 8%, about 7%, about 6%, about 5%, about 4%, about 3%, about 2% or about 1%. An embodiment wherein the surfactant is in an amount of about 0.5% by weight is preferred.

The surfactant can have a hydrophilic/lipophilic balance (HLB) value between about 1 and about 25 and a melting point between about 25° C. and about 70° C. The HLB characteristic of surfactants can be determined in accordance with “Physical Pharmacy: Physical Chemical Principles in the Pharmaceutical Sciences,” Fourth Edition, pp. 371-373, A. Martin, Ed., Lippincott Williams & Wilkins, Philadelphia (1993). Suitable surfactants include: glyceryl monocaprylate (e.g., Capmul® MCM), Pluronic® 10R5, Pluronic® 17R2, Pluronic® 17R4, Pluronic® 25R2, Pluronic® 25R4, Pluronic® 31R1, Pluronic® F 108, Pluronic® F 108 NF, Pluronic® F 108, Pluronic® F 108NF, Poloxamer 338, Pluronic® F 127, Pluronic® F 127 NF, Pluronic® F 127 NF 500 BHT Prill, Pluronic® F. 127 NF Prill, Poloxamer 407, Pluronic® F. 38, Pluronic® F. 38 Pastille, Pluronic® F. 68, Pluronic® F 68 LF Pastille, Pluronic® F 68 NF, Pluronic® F 68 NF Prill, Poloxamer 188, Pluronic® F. 68 Pastille, Pluronic® F. 77, Pluronic® F. 77 Micropastille, Pluronic® F. 87, Pluronic® F. 87 NF, Pluronic® F. 87 NF Prill, Poloxamer 237, Pluronic® F. 88, Pluronic® F. 88 Pastille, Pluronic® F. 98, Pluronic® L 10, Pluronic® L 101, Pluronic® L 121, Pluronic® L 31, Pluronic® L 35, Pluronic® L 43, Pluronic® L 61, Pluronic® L 62, Pluronic® L 62 LF, Pluronic® L 62D, Pluronic® L 64, Pluronic® L 81, Pluronic® L 92, Pluronic® N 3, Pluronic® P 103, Pluronic® P 104, Pluronic® P 105, Pluronic® P 123 Surfactant, Pluronic® P 65, Pluronic® P 84, Pluronic® P 85, Adogen® 464, Alkanol® 6112, Brij® 52, Brij® 93, Brij® S2, Brij® S, Brij® 58, Brij® C10, Brij® L4, Brij® 010, Brij® 010, BRIJ® 020, Brij® S10, Brij® S20, ethylenediamine tetrakis(ethoxylate-block-propoxylate) tetrol, ethylenediamine tetrakis(ethoxylate-block-propoxylate) tetrol, ethylenediamine tetrakis(propoxylate-block-ethoxylate) tetrol, IGEPAL® CA-210, IGEPAL® CA-520, IGEPAL® CA-720, IGEPAL® CO-520, IGEPAL® CO-630, IGEPAL® CO-720, IGEPAL® CO-890, IGEPAL® DM-970, MERPOL® DA, MERPOL® HCS, MERPOL® OJ, MERPOL® SE, MERPOL® SH, MERPOL® A, Poly(ethylene glycol) sorbitan tetraoleate, poly(ethylene glycol) sorbitol hexaoleate, poly(ethylene glycol) (12), poly(ethylene glycol) (18), polyethylene-block-poly(ethylene glycol), sorbitan monopalmitate, 2,4,7,9-tetramethyl-5-decyne-4,7-diol ethoxylate, Nonidet™ P-40, Triton™ N-101, Triton™ X-100, Triton™ X-114, Triton™ X-405, TWEEN® 20, TWEEN® 40, TWEEN® 60, TWEEN® 85, Zonyl® FS-300, or Zonyl® FSN. In one embodiment, the surfactant comprises Pluronic® F127, Tween® 80, Span® 80, IGEPAL®, Triton™ X-100, or Capmul® MCM.

Preferably, the surfactant is selected from the group consisting of: polyoxyethylene alkyl ethers, polyoxyethylene stearates, polyethylene glycols (PEG), poloxamers, poloxamines, sarcosine based surfactants, polysorbates, aliphatic alcohols, alkyl and aryl sulfates, alkyl and aryl polyether sulfonates and other sulfate surfactants, trimethyl ammonium based surfactants, lecithin and other phospholipids, bile salts, polyoxyethylene castor oil derivatives, polyoxyethylene sorbitan fatty acid esters, Sorbitan fatty acid esters, Sucrose fatty acid esters, alkyl glucopyranosides, alkyl maltopyranosides, glycerol fatty acid esters, Alkyl Benzene Sulphonic Acids, Alkyl Ether Carboxylic Acids, Alkyl and aryl Phosphate esters, Alkyl and aryl Sulphate esters, Alkyl and aryl Sulphonic acids, Alkyl Phenol Phosphates esters, Alkyl Phenol Sulphates esters, Alkyl and Aryl Phosphates, Alkyl Polysaccharides, Alkylamine Ethoxylates, Alkyl-Naphthalene Sulphonates formaldehyde condensates, Sulfosuccinates, lignosulfonates, Ceto-Oleyl Alcohol Ethoxylates, Condensed Naphthalene Sulphonates, Dialkyl and Alkyl Naphthalene Sulphonates, Di-alkyl Sulphosuccinates, Ethoxylated nonylphenols, Ethylene Glycol Esters, Fatty Alcohol Alkoxylates, Hydrogenated tallowalkylamines, Mono-alkyl Sulphosuccinamates, Nonyl Phenol Ethoxylates, Sodium Oleyl N-methyl Taurate, Tallowalkylamines, linear and branched dodecylbenzene sulfonic acids

Preferably, the surfactant is selected from the group consisting of: sodium lauryl sulfate, sodium stearyl sulfate, sodium cetyl sulfate, sodium cetostearyl sulfate, sodium docusate, sodium deoxycholate, N-lauroylsarcosine sodium salt, glyceryl monostearate, glycerol distearate glyceryl palmitostearate, glyceryl behenate, glyceryl caprylate, glyceryl oleate, benzalkonium chloride, CTAB, CTAC, Cetrimide, cetylpyridinium chloride, cetylpyridinium bromide, benzethonium chloride, PEG 40 stearate, PEG 100 stearate, poloxamer 188, poloxamer 338, poloxamer 407 polyoxyl 2 stearyl ether, polyoxyl 100 stearyl ether, polyoxyl 20 stearyl ether, polyoxyl 10 stearyl ether, polyoxyl 20 cetyl ether, polysorbate 20, polysorbate 40, polysorbate 60, polysorbate 61, polysorbate 65, polysorbate 80, polyoxyl 35 castor oil, polyoxyl 40 castor oil, polyoxyl 60 castor oil, polyoxyl 100 castor oil, polyoxyl 200 castor oil, polyoxyl 40 hydrogenated castor oil, polyoxyl 60 hydrogenated castor oil, polyoxyl 100 hydrogenated castor oil, polyoxyl 200 hydrogenated castor oil, cetostearyl alcohol, macrogel 15 hydroxystearate, sorbitan monopalmitate, sorbitan monostearate, sorbitan trioleate, Sucrose Palmitate, Sucrose Stearate, Sucrose Distearate, Sucrose laurate, Glycocholic acid, sodium Glycholate, Cholic Acid, Sodium Cholate, Sodium Deoxycholate, Deoxycholic acid, Sodium taurocholate, taurocholic acid, Sodium taurodeoxycholate, taurodeoxycholic acid, soy lecithin, phosphatidylcholine, phosphatidylethanolamine, phosphatidylserine, phosphatidylinositol, PEG4000, PEG6000, PEG8000, PEG10000, PEG20000, alkyl naphthalene sulfonate condensate/Lignosulfonate blend, Calcium Dodecylbenzene Sulfonate, Sodium Dodecylbenzene Sulfonate, Diisopropyl naphthalenesulphonate, erythritol distearate, Naphthalene Sulfonate Formaldehyde Condensate, nonylphenol ethoxylate (poe-30), Tristyrylphenol Ethoxylate, Polyoxyethylene (15) tallowalkylamines, sodium alkyl naphthalene sulfonate, sodium alkyl naphthalene sulfonate condensate, sodium alkylbenzene sulfonate, sodium isopropyl naphthalene sulfonate, Sodium Methyl Naphthalene Formaldehyde Sulfonate, sodium n-butyl naphthalene sulfonate, tridecyl alcohol ethoxylate (poe-18), Triethanolamine isodecanol phosphate ester, Triethanolamine tristyrylphosphate ester, Tristyrylphenol Ethoxylate Sulfate, Bis(2-hydroxyethyl)tallowalkylamines.

The surfactant system may include any surfactant as long as it is compatible with pharmaceutical applications. For example, suitable surfactants include, but are not limited to, polyoxyethylene sorbitan fatty acid esters (polysorbates such as polysorbate 20 or 80), poloxamers (such as LUTROL™ F68, F108 and F127 which are block copolymers of ethylene oxide and propylene oxide, sodium dodecylsulfate and/or sodium lauryl sulphate), sorbitan esters of fatty acids (SPAN), polyethoxylated castor oil and its derivatives, tocopheryl polyethylene glycol succinate, and polyvinyl alcohols. In certain embodiments, the surfactant system comprises an amount of surfactant that ranges from about 0.01% (w/v) to about 5% (w/v) surfactant. In other embodiments, the surfactant system comprises an amount of surfactant that ranges from about 0.1% (w/v) to about 3% (w/v) surfactant. In still other embodiments, the surfactant system comprises about 0.2% (w/v) surfactant. In still other embodiments, the surfactant system comprises about 0.4% (w/v) surfactant. In other embodiments, the surfactant system comprises polysorbate-80 (e.g., Tween-80). In still other embodiments, the surfactant system comprises 0.4% (w/v) polysorbate-80.

### Wax

When present within the composition, the fat/wax generally makes up between about 5% to about 40% by weight of the composition, more preferably between about 5% to about 30% by weight of the composition. Those percentages may, however, vary depending upon the number of additional materials contained within the composition. Thus, for example, the fat/wax may make up a larger percentage by weight of the a granulate that uses only a first material and one which includes both, for example, ethylcellulose and HPMC.

The fat/wax preferably used in the composition and coatings of the present invention, and indeed in the matrix or excipients in compositions and dosage forms, are hydrophobic and may be solid at room temperature (25° C.). Fats are fatty acid based compounds generally having a hydrophilic/lipophilic balance (HLB) of about 6 or less, more preferably 4 or less, and most preferably 2 or less, and also have a melting point which is preferably 30° C. or more, more preferably 40° C. and even more preferably 50° C. In one embodiment, the fat has an HLB of about 6 or less and a melting point of about 30° C. or more. In another embodiment, it has an HLB of about 4 or less and a melting point of about 40° C. or more. In another embodiment, the fat has an HLB of about 2 or less and a melting point of about 50° C. or more. Fats, including fatty acids and fatty esters in accordance with the present invention may be substituted or unsubstituted, saturated or unsaturated. However, generally they have a chain length of at least about 14. The esters in question may include fatty acid groups bound to alcohols, glycols, and in particularly preferred embodiment, glycerol. With regard to glyercols, for example, mono-, di-, and tri-fatty substituted glycerols are contemplated as are mixtures thereof. Thixotropic fats/waxes can also be used.

Suitable fat ingredients include, without limitation, glycerol fatty esters, fatty glyceride derivatives, waxes and fatty alcohols such as, for example, glycerol behenate (COMPRITOL®), glycerol palmitostearate (PRECIROL®), stearoyl macroglycerides (GELUCIRE®50/13). A particularly preferred material useful in accordance with the present invention is glycerol behenate.

Waxes include, without limitation, insect and animal waxes, vegetable waxes, mineral waxes, petroleum waxes, and synthetic waxes. Particularly preferred are beeswax, carnauba wax, condelilla wax, montan wax, ouricury wax, rice-bran wax, jojoba wax, microcrystalline wax, cetyl ester wax, anionic emulsifying wax, nonionic emulsifying wax and paraffin wax. In one embodiment, the fat/wax is a fatty acid ester of glycerol. In another, the fatty acid ester of glycerol is glycerol behenate, glyceryl dibehenate, and/or glyceryl behenate, such as COMPRITOL® 888ATO.

More preferably, these materials are also listed in one or more compendia, meaning they have been recognized for use in oral pharmaceutical products.

### Matrix Formulations

The present invention relates to the field of abuse and tamper resistant immediate and controlled release formulations comprising a drug-resin complex with one or more swellable, and non-swellable hydrophilic, non-swellable lipophilic and wax polymeric matrices followed by curing between 60° C. and 85° C. for 5 seconds to 300 seconds.

The present invention comprises, in addition to one or more active ingredients with abuse potential (A) optionally together with physiologically acceptable one or more high and/or low molecular weight water soluble polyethylene swellable polymer, (B) one or more non-swellable low molecular polyethylene glycol, and one or more waxes to formulate into abuse and tamper resistant dosage forms.

The kinetic drug release from matrix tablets follows the Fickian law of diffusion, which is driven by particle size of resinate, amount of polymer, degree of swelling and concentration gradient exist between tablet and surrounding media. Furthermore, according to this invention, the kinetic drug release from the tablet is modulated by a curing process between 60° C. and 85° C. for 5 seconds to 300 seconds.

In certain embodiments of the present invention, the sustained release formulation is achieved via a matrix optionally having a controlled release coating as set forth herein. The present invention may also utilize a sustained release matrix that affords in-vitro dissolution rates of the opioid analgesic and or antagonist within desired ranges and releases the opioid analgesic and/or antagonist in a pH-dependent or pH-independent manner.

A non-limiting list of suitable sustained-release materials which may be included in a sustained-release matrix according to the invention includes hydrophilic and/or hydrophobic materials, such as gums, cellulose ethers, acrylic resins, protein derived materials, waxes, shellac, and oils such as hydrogenated castor oil and hydrogenated vegetable oil. However, any pharmaceutically acceptable hydrophobic or hydrophilic sustained-release material which is capable of imparting sustained-release of the opioid analgesic may be used in accordance with the present invention. Preferred sustained-release polymers include alkylcelluloses such as ethylcellulose, acrylic and methacrylic acid polymers and copolymers; and cellulose ethers, especially hydroxyalkylcelluloses (especially hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methylmethacrylate), poly(methacrylic acid) (anhydride), polymethacrylate, polyacrylamide, poly(methacrylic acid anhydride), and glycidyl methacrylate copolymers. Certain preferred embodiments utilize mixtures of any of the foregoing sustained-release materials in the matrix of the invention.

Preferred embodiments are exemplified as follows:

TABLE 1
Molecular Weight of Polymers

| Polymer | Molecular Weight | Drug to Excipient Ratio |
|---|---|---|
| Poly(vinyl) Pyrollidone | 50,000-60,000 | 4:1 to 4:4 |
| Poly(vinyl) Alcohol | 88000 | 4:0.1 to 4:3.6 |
| Poly(ethylene oxide) (PEO) | 200,000 | 4:1 to 4:3.6 |
| HPMC | 34,500 | 4:0.1 to 4:3.6 |
| Cellulose acetate | | 4:0.1 to 4:3 |
| EthylCellulose EC 20 cP | | 4:1 to 4:4 |

The matrix also may include a binder. In such embodiments, the binder preferably contributes to the sustained-release of the oxycodone or pharmaceutically acceptable salt thereof from the sustained-release matrix.

If an additional hydrophobic binder material is included, it is preferably selected from natural and synthetic waxes, fatty acids, fatty alcohols, and mixtures of the same. Examples include beeswax, carnauba wax, stearic acid and stearyl alcohol. This list is not meant to be exclusive. In certain preferred embodiments, a combination of two or more hydrophobic binder materials are included in the matrix formulations.

Preferred hydrophobic binder materials which may be used in accordance with the present invention include digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and polyalkylene glycols. Hydrocarbons having a melting point of between 25° and 90° C. are preferred. Of the long-chain hydrocarbon binder materials, fatty (aliphatic) alcohols are preferred in certain embodiments. The oral dosage form may contain up to 80% (by weight) of at least one digestible, long chain hydrocarbon.

In certain embodiments, the hydrophobic binder material may comprise natural or synthetic waxes, fatty alcohols (such as lauryl, myristyl, stearyl, cetyl or preferably cetostearyl alcohol), fatty acids, including but not limited to fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30 to about 100° C. In certain preferred embodiments, the dosage form comprises a sustained release matrix comprising an opioid analgesic; opioid antagonist; one or more aversive agents; and at least one water soluble hydroxyalkyl cellulose, at least one C₁₂-C₃₆, preferably C₁₄-C₂₂, aliphatic alcohol and, optionally, at least one polyalkylene glycol. The hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present oral dosage form may be determined, inter alia, by the precise rate of opioid analgesic release required. The aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of the aliphatic alcohol in the present oral dosage form may be determined, as above, by the precise rate of opioid analgesic release required. It may also depend on whether at least one polyalkylene glycol is present in or absent from the oral dosage form. In the absence of at least one polyalkylene glycol, the oral dosage form preferably contains between about 20% and about 50% (by wt) of the aliphatic alcohol. When a polyalkylene glycol is present in the oral dosage form, then the combined weight of the aliphatic alcohol and the polyalkylene glycol preferably constitutes between about 20% and about 50% (by wt) of the total dosage form.

In one preferred embodiment, the ratio of, e.g., the at least one hydroxyalkyl cellulose or acrylic resin to the at least one aliphatic alcohol/polyalkylene glycol determines, to a considerable extent, the release rate of the opioid analgesic from the formulation. In certain embodiments, a ratio of the hydroxyalkyl cellulose to the aliphatic alcohol/polyalkylene glycol of between 1:1 and 1:4 is preferred, with a ratio of between 1:2 and 1:3 being particularly preferred.

In certain embodiments, the polyalkylene glycol may be, for example, polypropylene glycol, or polyethylene glycol which is preferred. The average molecular weight of the at least one polyalkylene glycol is preferably between 1,000 and 15,000, especially between 1,500 and 12,000.

Another suitable sustained-release matrix comprises an alkylcellulose (especially ethylcellulose), a C₁₂ to C₃₆ aliphatic alcohol and, optionally, a polyalkylene glycol.

In addition to the above ingredients, a sustained-release matrix may also contain suitable quantities of other materials, e.g., diluents, lubricants, binders, granulating aids and glidants that are conventional in the pharmaceutical art.

A sustained-release matrix can be prepared by, e.g., melt-granulation or melt-extrusion techniques. Generally, melt-granulation techniques involve melting a normally solid hydrophobic binder material, e.g., a wax, and incorporating a powdered drug therein. To obtain a sustained release dosage form, it may be necessary to incorporate a hydrophobic sustained-release material, e.g. ethylcellulose or a water-insoluble acrylic polymer, into the molten wax hydrophobic binder material.

The additional hydrophobic binder material may comprise one or more water-insoluble wax-like thermoplastic substances possibly mixed with one or more wax-like thermoplastic substances being less hydrophobic than said one or more water-insoluble wax-like substances. In order to achieve sustained release, the individual wax-like substances in the formulation should be substantially non-degradable and insoluble in gastrointestinal fluids during the initial release phases. Useful water-insoluble wax-like binder substances may be those with a water-solubility that is lower than about 1:5,000 (w/w).

The preparation of a suitable melt-extruded matrix according to the present invention may, for example, include the steps of blending the opioid analgesic, opioid antagonist, and at least one aversive agent, together with a sustained release material and preferably a binder material to obtain a homogeneous mixture. The homogeneous mixture is then heated to a temperature sufficient to at least soften the mixture sufficiently to extrude the same. The resulting homogeneous mixture is then extruded, e.g., using a twin-screw extruder, to form strands. The extrudate is preferably cooled and cut into multiparticulates by any means known in the art. The matrix multiparticulates are then divided into unit doses. The extrudate preferably has a diameter of from about 0.1 to about 5 mm and provides sustained release of the oxycodone or pharmaceutically acceptable salt thereof for a time period of at least about 24 hours.

An optional process for preparing the melt extruded formulations of the present invention includes directly metering into an extruder a hydrophobic sustained release material, the opioid analgesic, opioid antagonist, one or more aversive agents, and an optional binder material; heating the homogenous mixture; extruding the homogenous mixture to thereby form strands; cooling the strands containing the homogeneous mixture; cutting the strands into matrix multiparticulates having a size from about 0.1 mm to about 12 mm; and dividing said particles into unit doses. In this aspect of the invention, a relatively continuous manufacturing procedure is realized.

Plasticizers, such as those described above, may be included in melt-extruded matrices. The plasticizer is preferably included as from about 0.1 to about 30% by weight of the matrix. Other pharmaceutical excipients, e.g., talc, mono or poly saccharides, lubricants and the like may be included in the sustained release matrices of the present invention as desired. The amounts included will depend upon the desired characteristic to be achieved.

The diameter of the extruder aperture or exit port can be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt extruded matrix multiparticulate system can be, for example, in the form of granules, spheroids or pellets depending upon the extruder exit orifice. For purposes of the present invention, the terms “melt-extruded matrix multiparticulate(s)” and “melt-extruded matrix multiparticulate system(s)” and “melt-extruded matrix particles” shall refer to a plurality of units, preferably within a range of similar size and/or shape and containing one or more active agents and one or more excipients, preferably including a hydrophobic sustained release material as described herein. Preferably the melt-extruded matrix multiparticulates will be of a range of from about 0.1 to about 12 mm in length and have a diameter of from about 0.1 to about 5 mm. In addition, it is to be understood that the melt-extruded matrix multiparticulates can be any geometrical shape within this size range. In certain embodiments, the extrudate may simply be cut into desired lengths and divided into unit doses of the therapeutically active agent without the need of a spheronization step.

In one preferred embodiment, oral dosage forms are prepared that include an effective amount of melt-extruded matrix multiparticulates within a capsule. For example, a plurality of the melt-extruded matrix multiparticulates may be placed in a gelatin capsule in an amount sufficient to provide an effective sustained release dose when ingested and contacted by gastrointestinal fluid.

In another embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are described in Remington's Pharmaceutical Sciences, (Arthur Osol, editor), 1553-1593 (1980).

Optionally, the sustained-release matrix multiparticulate systems, tablets, or capsules can be coated with a sustained release coating such as the sustained release coatings described herein. Such coatings preferably include a sufficient amount of hydrophobic and/or hydrophilic sustained-release material to obtain a weight gain level from about 2 to about 25 percent, although the overcoat may be greater depending upon, e.g., the desired release rate. In certain embodiments, a sustained release coating is applied to the sustained release spheroids, granules, or matrix multiparticulates. In such embodiments, the sustained-release coating may include a water insoluble material such as (a) a wax, either alone or in admixture with a fatty alcohol; or (b) shellac or zein. The coating is preferably derived from an aqueous dispersion of the hydrophobic sustained release material.

In other preferred embodiments of the present invention, the sustained release material comprising the sustained-release coating is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art as fully polymerized copolymers of acrylic and methacrylic acid esters with a low content of quaternary ammonium groups. In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Rohm GMBH and Co. Kg Darmstadt, Germany. There are several different types of Eudragit®. For example, Eudragit E is an example of a methacrylic acid copolymer which swells and dissolves in acidic media. Eudragit L is a methacrylic acid copolymer which does not swell at about pH<5.7 and is soluble at about pH>6. Eudragit S does not swell at about pH<6.5 and is soluble at about pH>7. Eudragit RL and Eudragit RS are water swellable, and the amount of water absorbed by these polymers is pH-dependent; however, dosage forms coated with Eudragit RL and RS are pH-independent.

In certain preferred embodiments, the acrylic coating comprises a mixture of two acrylic resin lacquers commercially available under the Tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Methyl citrate is an especially preferred plasticizer for the aqueous dispersions of ethyl cellulose of the present invention.

In addition to the above ingredients, the spheroids, granules, or matrix multiparticulates may also contain suitable quantities of other materials, e.g., diluents, lubricants, binders, granulating aids, and glidants that are conventional in the pharmaceutical art in amounts up to about 50% by weight of the formulation if desired. The quantities of these additional materials will be sufficient to provide the desired effect to the desired formulation.

In one embodiment, at least one active agent in solubility-improved form is incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or “matrix” that entraps the solubility-improved form of the active agent. The aqueous-swollen matrix gradually erodes, swells, disintegrates or dissolves in the environment of use, thereby controlling the release of the active agent to the environment of use.

The erodible polymeric matrix into which the active agent is incorporated may generally be described as a set of excipients that are mixed with the solubility-improved form following its formation that, when contacted with the aqueous environment of use imbibes water and forms a water-swollen gel or “matrix” that entraps the drug form. Drug release may occur by a variety of mechanisms: the matrix may disintegrate or dissolve from around particles or granules of the drug in solubility-improved form; or the drug may dissolve in the imbibed aqueous solution and diffuse from the tablet, beads or granules of the device. A key ingredient of this water-swollen matrix is the water-swellable, erodible, or soluble polymer, which may generally be described as an osmopolymer, hydrogel or water-swellable polymer. Such polymers may be linear, branched, or crosslinked. They may be homopolymers or copolymers. Although they may be synthetic polymers derived from vinyl, acrylate, methacrylate, urethane, ester and oxide monomers, they are most preferably derivatives of naturally occurring polymers such as polysaccharides or proteins.

Such materials include naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonium alginate, sodium, potassium or calcium alginate, propylene glycol alginate;

gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

A preferred class of cellulosics for the erodible matrix comprises aqueous-soluble and aqueous-erodible cellulosics such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL series E5, E15LV, E50LV and K100LY, while high viscosity HPMC polymers include E4MCR, E10MCR, K4M, K15M and K100M; especially preferred in this group are the METHOCEL (Trademark) K series. Other commercially available types of HPMC include the Shin Etsu METOLOSE 90SH series.

Although the primary role of the erodible matrix material is to control the rate of release of the active agent in solubility-improved form to the environment of use, the inventors have found that the choice of matrix material can have a large effect on the maximum drug concentration attained by the device as well as the maintenance of a high drug concentration. In one embodiment, the matrix material is a concentration-enhancing polymer, as defined herein below.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl) methacrylate chloride.

The erodible matrix polymer may contain a wide variety of the same types of additives and excipients known in the pharmaceutical arts, including osmopolymers, osmagens, solubility-enhancing or -retarding agents and excipients that promote stability or processing of the device.

The formulation may comprise an excipient that is a swellable material such as a hydrogel in amounts that can swell and expand. Examples of swellable materials include polyethylene oxide, hydrophilic polymers that are lightly cross-linked, such cross-links being formed by covalent or ionic bond, which interact with water and aqueous biological fluids and swell or expand to some equilibrium state. Swellable materials such as hydrogels exhibit the ability to swell in water and retain a significant fraction of water within its structure, and when cross-linked they will not dissolve in the water. Swellable polymers can swell or expand to a very high degree, exhibiting a 2 to 50 fold volume increase. Specific examples of hydrophilic polymeric materials include poly(hydroxyalkyl methacrylate), poly(N-vinyl-2-pyrrolidone), anionic and cationic hydrogels, polyelectrolyte complexes, poly(vinyl alcohol) having a low acetate residual and cross-linked with glyoxal, formaldehyde, or glutaraldehyde, methyl cellulose cross-linked with dialdehyde, a mixture of cross-linked agar and carboxymethyl cellulose, a water insoluble, water-swellable copolymer produced by forming a dispersion of finely divided copolymer of maleic anhydride with styrene, ethylene, propylene, butylene, or isobutylene cross-linked with from 0.001 to about 0.5 moles of a polyunsaturated cross-linking agent per mole of maleic anhydride in the copolymer, water-swellable polymers of N-vinyl lactams, cross-linked polyethylene oxides, and the like. Other examples of swellable materials include hydrogels exhibiting a cross-linking of 0.05 to 60%, hydrophilic hydrogels known as Carbopol acidic carboxy polymer, Cyanamer™ polyacrylamides, cross-linked water-swellable indene-maleic anhydride polymers, Good-Rite™ polyacrylic acid, starch graft copolymers, Aqua-Keeps™ acrylate polymer, diester cross-linked polyglucan, and the like.

The formulations may comprise additives such as polyethylene oxide polymers, polyethylene glycol polymers, cellulose ether polymers, cellulose ester polymers, homo- and copolymers of acrylic acid cross-linked with a polyalkenyl polyether, poly(meth)acrylates, homopolymers (e.g., polymers of acrylic acid crosslinked with allyl sucrose or allyl pentaerythritol), copolymers (e.g., polymers of acrylic acid and C₁₀-C₃₀ alkyl acrylate crosslinked with allyl pentaerythritol), interpolymers (e.g., a homopolymer or copolymer that contains a block copolymer of polyethylene glycol and a long chain alkyl acid ester), disintegrants, ion exchange resins, polymers reactive to intestinal bacterial flora (e.g., polysaccharides such as guar gum, inulin obtained from plant or chitosan and chondroitin sulphate obtained from animals or alginates from algae or dextran from microbial origin) and pharmaceutical resins.

### Osmotic Dosage Forms

Sustained release dosage forms according to the present invention may also be prepared as osmotic dosage formulations. The osmotic dosage forms preferably include a bilayer core comprising a drug layer (containing the active agent, such as an opioid) and a delivery c: push layer (which may contain the opioid antagonist and/or one or more aversive agents), wherein the bilayer core is surrounded by a semipermeable wall and optionally having at least one passageway disposed therein.

In further embodiments, the present invention discloses a novel dosage form for delivering at least one drug susceptible to abuse. Further, it releases drug in controlled rate over period of time at variable rate across the pH range from 1.0 to 8.0. The therapeutic agent and agents may be present in ion exchange form. The osmotic drug delivery system including a gelling agent with one or more salts and inorganic acids to provide a particular viscosity, and enhance the solubility power of the hydrated core in order to release the therapeutic agent in molecular form through orifice at predefined release rate. Additionally, in the present invention at least one therapeutic agent may be present in an ion exchange complex form, this renders abuse-capable matrix system with one or more gelling agents that difficult to separate the opioid and others prone to abuse from the finished drug dosage form. The osmotic drug delivery system provides therapeutically effective steady state plasma concentration for, for example, 12 to 24 hours when administered twice or once per day.

As used herein, “osmotic delivery system” or “osmotic controlled release oral delivery system” or “OROS” refers to a controlled release oral drug delivery system in the form of a tablet. The tablet has a rigid water-permeable jacket with one or more laser drilled small holes. As the tablet passes through the body, the osmotic pressure of water entering the tablet pushes the active drug through the opening in the tablet.

The expression “passageway” as used for the purpose of this invention, includes aperture, orifice, bore, pore, porous element through which the opioid analgesic (with or without the antagonist) can be pumped, diffuse or migrate through a fiber, capillary tube, porous overlay, porous insert, microporous member, or porous composition. The passageway can also include a compound that erodes or is leached from the wall in the fluid environment of use to produce at least one passageway. Representative compounds for forming a passageway include erodible poly(glycolic) acid, or poly(lactic) acid in the wall; a gelatinous filament; a water-removable poly(vinyl alcohol); leachable compounds such as fluid-removable pore-forming polysaccharides, acids, salts or oxides. A passageway can be formed by leaching a compound from the wall, such as sorbitol, sucrose, lactose, maltose, or fructose, to form a sustained-release dimensional pore-passageway. The passageway can have any shape, such as round, triangular, square and elliptical, for assisting in the sustained metered release of opioid analgesic from the dosage form. The dosage form can be manufactured with one or more passageways in spaced-apart relation on one or more surfaces of the dosage form.

In certain embodiments, the bilayer core comprises a drug layer and a displacement or push layer. In certain embodiments the drug layer may also comprise at least one polymer hydrogel. The polymer hydrogel may have an average molecular weight of between about 500 and about 6,000,000. Examples of polymer hydrogels include but are not limited to a maltodextrin polymer comprises a 500 to 1,250,000 number-average molecular weight; a poly(alkylene oxide) represented by, e.g., a poly(ethylene oxide) and a poly(propylene oxide) having a 50,000 to 750,000 weight-average molecular weight, and more specifically represented by a poly(ethylene oxide) of at least one of 100,000, 200,000, 300,000 or 400,000 weight-average molecular weights; an alkali carboxyalkylcellulose, wherein the alkali is sodium or potassium, the alkyl is methyl, ethyl, propyl, or butyl of 10,000 to 175,000 weight-average molecular weight; and a copolymer of ethylene-acrylic acid, including methacrylic and ethacrylic acid of 10,000 to 500,000 number-average molecular weight.

In certain embodiments of the present invention, the delivery or push layer comprises an osmopolymer. Examples of an osmopolymer include but are not limited to a member selected from the group consisting of a polyalkylene oxide and a carboxyalkylcellulose. The polyalkylene oxide possesses a 1,000,000 to 10,000,000 weight-average molecular weight. The polyalkylene oxide may be a member selected from the group consisting of polymethylene oxide, polyethylene oxide, polypropylene oxide, polyethylene oxide having a 1,000,000 average molecular weight, polyethylene oxide comprising a 5,000,000 average molecular weight, polyethylene oxide comprising a 7,000,000 average molecular weight, cross-linked polymethylene oxide possessing a 1,000,000 average molecular weight, and polypropylene oxide of 1,200,000 average molecular weight. Typical osmopolymer carboxyalkylcellulose comprises a member selected from the group consisting of alkali carboxyalkylcellulose, sodium carboxymethylcellulose, potassium carboxymethylcellulose, sodium carboxyethylcellulose, lithium carboxymethylcellulose, sodium carboxyethylcellulose, carboxyalkylhydroxyalkylcellulose, carboxymethylhydroxyethyl cellulose, carboxyethylhydroxyethylcellulose and carboxymethylhydroxypropylcellulose. The osmopolymers used for the displacement layer exhibit an osmotic pressure gradient across the semipermeable wall. The osmopolymers imbibe fluid into dosage form, thereby swelling and expanding as an osmotic hydrogel (also known as osmogel), whereby they push the contents of the drug layer from the osmotic dosage form.

The push layer may also include one or more osmotically effective compounds also known as osmagents and as osmotically effective solutes. They imbibe an environmental fluid, for example, from the gastrointestinal tract, into dosage form and contribute to the delivery kinetics of the displacement layer. Examples of osmotically active compounds comprise a member selected from the group consisting of osmotic salts and osmotic carbohydrates. Examples of specific osmagents include but are not limited to sodium chloride, potassium chloride, magnesium sulfate, lithium phosphate, lithium chloride, sodium phosphate, potassium sulfate, sodium sulfate, potassium phosphate, glucose, fructose and maltose.

The push layer may optionally include a hydroxypropylalkylcellulose possessing a 9,000 to 450,000 number-average molecular weight. The hydroxypropylalkylcellulose is represented by a member selected from the group consisting of hydroxypropylmethylcellulose, hydroxypropylethylcellulose, hydroxy propyl isopropyl cellulose, hydroxypropylbutylcellulose, and hydroxypropylpentylcellulose.

In certain alternative embodiments, the dosage form comprises a substantially homogenous core comprising active agent, a pharmaceutically acceptable polymer (e.g., polyethylene oxide), optionally a disintegrant (e.g., polyvinylpyrrolidone), optionally an absorption enhancer (e.g., a fatty acid, a surfactant, a chelating agent, a bile salt, etc.). The substantially homogenous core is surrounded by a semipermeable wall having a passageway (as defined above) for the release of the active agent.

In certain embodiments, the semipermeable wall comprises a member selected from the group consisting of a cellulose ester polymer, a cellulose ether polymer and a cellulose ester-ether polymer. Representative wall polymers comprise a member selected from the group consisting of cellulose acylate, cellulose diacylate, cellulose triacylate, cellulose acetate, cellulose diacetate, cellulose triacetate, mono-, di- and tricellulose alkenylates, and mono-, di- and tricellulose alkinylates. The poly(cellulose) used for the present invention comprises a number-average molecular weight of 20,000 to 7,500,000.

In certain embodiments, preferably the semipermeable wall is nontoxic, inert, and it, maintains its physical and chemical integrity during the dispensing life of the drug. In certain embodiments, the dosage form comprises a binder. An example of a binder includes, but is not limited to a therapeutically acceptable vinyl polymer having a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinyl-pyrrolidone copolymers with a member selected from the group consisting of vinyl acetate, vinyl alcohol, vinyl chloride, vinyl fluoride, vinyl butyrate, vinyl laureate, and vinyl stearate. Other binders include for example, acacia, starch, gelatin, and hydroxypropylalkylcellulose of 9,200 to 250,000 average molecular weight.

In certain embodiments, the dosage form comprises a lubricant, which may be used during the manufacture of the dosage form to prevent sticking to die wall or punch faces. Examples of lubricants include but are not limited to magnesium stearate, sodium stearate, stearic acid, calcium stearate, magnesium oleate, oleic acid, potassium oleate, caprylic acid, sodium stearyl fumarate, and magnesium palmitate.

### Solvents

Pharmaceutically acceptable solvents may be an aqueous or organic solvent such as, for example, methanol, ethanol, isopropranol, ethylene glycol, acetone, or mixtures thereof. In other embodiments, pharmaceutically acceptable solvents may include, but are not limited to, polypropylene glycol, polypropylene glycol, polyethylene glycol, for example, polyethylene glycol 600, polyethylene glycol 900, polyethylene glycol 540, polyethylene glycol 1450, polyethylene glycol 6000, polyethylene glycol 8000, and the like; pharmaceutically acceptable alcohols that are liquids at about room temperature, for example, propylene glycol, ethanol, 2-(2-ethoxyethoxy)ethanol, benzyl alcohol, glycerol, polyethylene glycol 200, polyethylene glycol 300, polyethylene glycol 400 and the like, polyoxyethylene castor oil derivatives, for example, polyoxyethyleneglycerol triricinoleate or polyoxyl 35 castor oil, polyoxyethyleneglycerol oxystearate, RH 40 (polyethyleneglycol 40 hydrogenated castor oil) or RH 60 (polyethyleneglycol 60 hydrogenated castor oil), and the like, saturated polyglycolized glycerides; polyoxyethylene alkyl ethers, for example, cetomacrogol 1000 and the like; polyoxyethylene stearates, for example, PEG-6 stearate, PEG-8 stearate, polyoxyl 40 stearate NE, polyoxyethyl 50 stearate NF, PEG-12 stearate, PEG-20 stearate, PEG-100 stearate, PEG-12 distearate, PEG-32 distearate, PEG-150 distearate and the like; ethyl oleate, isopropyl palmitate, isopropyl myristate and the like; dimethyl isosorbide; N-methylpyrrolidinone; paraffin; cholesterol; lecithin; suppository bases; pharmaceutically acceptable waxes, for example, carnauba wax, yellow wax, white wax, microcrystalline wax, emulsifying wax and the like; pharmaceutically acceptable silicon fluids; sorbitan fatty acid esters such as sorbitan laurate, sorbitan oleate, sorbitan palmitate, sorbitan stearate and the like; pharmaceutically acceptable saturated fats or pharmaceutically acceptable saturated oils, for example, hydrogenated castor oil (glyceryl-tris-12-hydroxystearate), cetyl esters wax (a mixture of primarily C₁₄-C₁₈ saturated esters of C₁₄-C₁₈ saturated fatty acids having a melting range of about 43-47° C.), glyceryl monostearate and the like.

The hydrophilic solvent may comprise an alcohol, ketone, hydrocarbon or polar aprotic solvent, and mixtures and aqueous solutions thereof. Hydrophilic solvents may also be utilized as solubilizers include, for example, alcohols, for example, water miscible alcohols, such as, ethanol or glycerol; glycols such as 1,2-propylene glycol; polyols such as a polyalkylene glycol, for example, polyethylene glycol. Alternatively, hydrophilic solvents may include N-alkylpyrolidones such as N-methylpyrrolidone, triethylcitrate, dimethyl isosorbide, caprylic acid, or propylene carbonate. In still other embodiments, the solubilizer may be a nitrogen-containing solvent such as, for example, acetonitrile, dimethylformamide, dimethylacetamide, N-alkylpyrrolidone, N-hydroxyalkylpyrrolidone, N-alkylpiperidone, N-alkylcaprolactam, and mixtures thereof wherein alkyl may be a C₁₋₁₂ branched or straight chain alkyl. In particular embodiments, nitrogen-containing solvents may include N-methyl 2-pyrrolidone, N-ethyl 2-pyrrolidone, or a mixture thereof. Alternatively, the nitrogen-containing solvent may be in the form of a polymer such as polyvinylpyrrolidone.

The invention will be illustrated in more detail with reference to the following Examples, but it should be understood that the present invention is not deemed to be limited thereto.

### EXAMPLES

### Example 1. Self-Emulsified Abuse and Tamper Resistant Liquid Filled Dosage Forms

### Composition 1:

| S. No. | Ingredient (s) | % w/w |
|---|---|---|
| 1. | Oxycodone Base | 8.16 |
| 2. | Ethylcellulose (40 cP) | 10.20 |
| 3. | Oleic Acid | 61.22 |
| 4. | PEG-8 Caprylic/Capric Glycerides | 10.22 |
| | (Labrasol) | |
| 5. | Polyoxyl 40 Hydrogenated Castor Oil | 10.20 |
| | (Cremophor RH) | |
| | Total | 100.00 |

### Composition 2:

| S. No. | Ingredient (s) | % w/w |
|---|---|---|
| 1. | Oxycodone Base | 9.07 |
| 2. | Ethylcellulose (40 cP) | 9.07 |
| 3. | Oleic Acid | 62.97 |
| 4. | PEG-8 Caprylic/Capric Glycerides | 6.30 |
| | (Labrasol) | |
| 5. | Polyoxyl 40 Hydrogenated Castor Oil | 12.59 |
| | (Cremophor RH) | |
| | Total | 100.00 |

### Composition 3:

| S. No. | Ingredient (s) | % w/w |
|---|---|---|
| 1. | Oxycodone Base | 9.07 |
| 2. | Ethylcellulose (40 cP) | 2.10 |
| 3. | Oleic Acid | 62.97 |
| 4. | PEG-8 Caprylic/Capric Glycerides | 13.27 |
| | (Labrasol) | |
| 5. | Polyoxyl 40 Hydrogenated Castor Oil | 12.59 |
| | (Cremophor RH) | |
| | Total | 100.00 |

### Composition 4:

The fill material of Example 1 was encapsulated into 20 oblong soft gelatin capsules using Bochang rotary die encapsulator and 200 bloom, limed bone, Type B, gelatin plasticized with polyols, using conventional methods (see, e.g., Wilkinson et al., “Softgels: Manufacturing considerations,” Drugs and the Pharmaceutical Sciences, 41 (Specialized Drug Delivery Systems), P. Tyle, Ed. (Marcel Dekker, Inc., New York, 1990) pp. 409-449; Horn et al., “Capsules, Soft,” Encyclopedia of Pharmaceutical Technology, Vol 2, J. Swarbrick and J. C. Boylan, eds. (Marcel Dekker, Inc., New York, 1990) pp 260-284; Patel et al., Manufacturing Chemist, 60(7): 26-28 (1989); Patel et al., Manufacturing Chemist, 60(8): 47-49 (1989); Jimerson et al., Drug Development and Industrial Pharmacy, 12(8-9): 1133-1144 (1986); and Ebert, Pharmaceutical Technology, 1(5):44-50 (1977)).

| S. No. | Ingredient (s) | % w/w |
|---|---|---|
| 1. | Gelatin Type B 200 bloom | 41.00 |
| 2. | Glycerin | 11.00 |
| 3. | Sorbitol Special | 11.00 |
| 4. | Purified Water, USP | 37.00 |
| | Total | 100.00 |

### Manufacturing Procedure:

Oxycodone Controlled Release Capsules, 40 mg was prepared by dissolving ethylcellulose in oleic acid at >80° C. followed by mixing labrasol and cremophor. Oxycodone base was dissolved in Oleic acid-Ethylcellulose matrix under continuous mixing and heating. The resultant clear viscous liquid was filled in hard gelatin capsules.

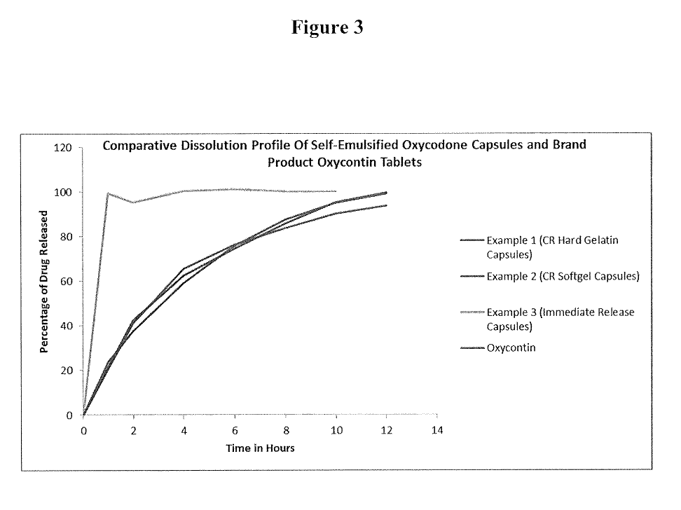

As shown in FIG. 3, Self-emulsified abuse and tamper resistant Oxycodone Capsules, 40 mg dissolution evaluated in pH 1.2 simulated gastric fluid (SGF) in a dissolution apparatus II (paddle) at 100 rpm and a volume of 900 ml maintained at a temperature of 37.0° C. (+0.5° C.), followed by HPLC method.

### Example 2. Abuse and Tamper Resistant Solid Dosage Forms Containing Therapeutic Agent in Complex Form with Ion-Exchange Resin

### Composition 5

| S. No. | Ingredient (s) | % w/w |
|---|---|---|
| 1. | Oxycodone HCl | 6.66-13.33 |
| 2. | Amberlite IRP 69 | 19.99-39.99 |
| 3. | Polyvinyl Alcohol | 1.5-5.50 |
| 4. | Polyethylene Glycol 600 | 1.00-5.00 |
| 6. | Microcrystalline Cellulose Powder | 69.75-29.18 |
| 7. | Colloidal Silicondioxide | 0.1-2.00 |
| 6. | Stearic Acid | 1.00-5.00 |
| | Total | 100.00 |

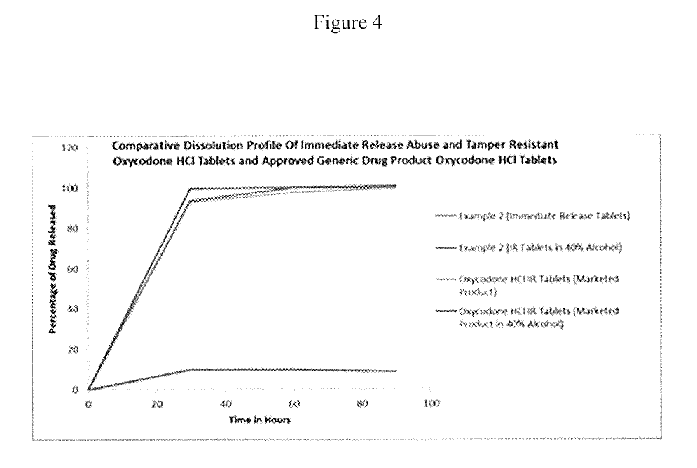

As shown in FIG. 4, Abuse and tamper resistant immediate release solid dosage form containing Oxycodone HCl in complex form with Amberlite IRP 69 was prepared by direct mixing with polyvinyl alcohol, polyethylene glycol 600, microcrystalline cellulose powder, colloidal silicon dioxide and stearic acid followed by compression. The resultant compressed tablets melted at 60-80° C. for 10 minutes then cooled to room temperature. The resultant tablets were cooled and evaluated for drug release rate in simulated gastric fluid at pH 1.2. The immediate release abuse and tamper resistant tablets released drug more than 90% in 30 minutes and less than 10% in 40% ethanol dissolution media.

### Composition 6

| S. No. | Ingredient | % w/w | Mg/tab |
|---|---|---|---|
| 1. | Oxycodone Resinate Eq. to | 41.11 | 185.00 |
| | Oxycodone HCl 40 mg | | |
| 2. | Polyethylene Oxide NE-80 | 41.11 | 185.00 |
| 3. | Carnauba Wax fine Powder | 7.78 | 35.00 |
| 4. | Polyethylene Glycol 8000 | 8.89 | 40.00 |
| 5. | Stearic Acid | 1.11 | 5.00 |
| | Total | 100.00 | 450.00 |

Manufacturing Procedure:
1. Blend all materials and screened through #30 mesh
2. Compress into tablets using appropriate punches
3. Cure the tablets between 40-50° C. under Infra Red (IR) lamp for 10-30 minutes.
4. Coat the tablets with pH independent polymer Ethylcellulose.

### Composition 7 (Controlled Release Abuse and Tamper Resistant Tablets)

| S. No. | Ingredient (s) | % w/w |
|---|---|---|
| 1. | Oxycodone HCl | 6.66-13.33 |
| 2. | Amberlite IRP 69 | 19.99-39.99 |
| 3. | Xanthan Gum | 10.00-30.00 |
| 4. | Polyethylene Glycol 600 | 1.00-5.00 |
| 5. | Carnauba Wax | 1.00-5.00 |
| 6. | Microcrystalline Cellulose Powder | 60.25-5.68 |
| 7. | Colloidal Silicondioxide | 0.10-1.00 |
| 6. | Stearic Acid | 1.00-5.00 |
| | Total | 100.00 |

### Composition 8 (Controlled Release Elementary Osmotic Oral Abuse and Tamper Resistant Tablets)

| S. No. | Ingredient (s) | % w/w |
|---|---|---|
| 1. | Oxycodone HCl | 6.66-13.33 |
| 2. | Amberlite IRP 69 | 19.99-39.99 |
| 3. | Polyethylene Oxide (NE 80) | 10.00-30.00 |
| 6. | Microcrystalline Cellulose Powder | 57.25-5.68 |
| 7. | Colloidal Silicondioxide | 0.10-1.00 |
| 6. | Stearic Acid | 1.00-5.00 |
| 7. | Sodium Chloride | 5.00 |
| | Total | 100.00 |

### Manufacturing Procedure:

Oxycodone Controlled Release Capsules, 40 mg was prepared by mixing all ingredients and passed through 40 screen then compressed into tablets at weight of 300 mg using 10 mm round punches. The compressed tablets cured at 70° C. for 200 seconds.

### Example 3. Abuse and Tamper Resistant Solid Dosage Forms Containing Therapeutic Agent in Complex Form with Ion-Exchange Resin

### Composition 9

| S. No | Ingredients | % w/w | mg/unit |
|---|---|---|---|
| | Layer 1 (Drug Layer) | | |
| 1. | Oxycodone Resinate Eq. to | 39.87 | 138.75 |
| | Oxycodone HCl 30 mg | | |
| 2. | Polyethylene Oxide N80 (WSR N80) | 57.65 | 200.61 |
| 3. | Povidone K29/32 | 1.72 | 6.00 |
| 4. | Succinic Acid | 0.72 | 2.52 |
| 5. | Magnesium stearate | 0.02 | 0.06 |
| 6. | Butylated Hydroxy Toluene | 0.02 | 0.06 |
| 7. | IPA | q.s | q.s |
| 8. | P Water | q.s | q.s |
| | | | 348.00 |
| | Layer 2 (Push Layer) | | |
| 9. | Polyethylene Oxide WSR Coagulant | 47.36 | 225.03 |
| | (WSR N-60K) | | |
| 10. | Sodium Chloride | 12.85 | 104.40 |
| 11. | Povidone K29/32 | 3.21 | 15.00 |
| 12. | Magnesium stearate | 0.16 | 0.50 |
| 13. | Butylated Hydroxy Toluene | 0.04 | 0.07 |
| 14. | Yellow Iron Oxide | 0.64 | 3.00 |
| 15. | IPA | q.s | q.s |
| 16. | P Water | q.s | q.s |
| | Total | 100.00 | 348.00 |
| | Total Tablet weight | | 696.00 |

### Example 4. Immediate Release—Abuse and Tamper Resistant Solid Dosage Form Containing Therapeutic Agent in Microencapsulated Form

Composition 10

Intragranular Part:

| | |
|---|---|
| Active | 30-40% |
| Silicified MCC | 10-40% |

Granulating Fluid:

| | |
|---|---|
| Precirol ATO | 0-10% |
| Compritol 888 ATO | 0-10% |
| PVP K 29/30 | 0-20% |
| Ethanol | q.s. |

Extragranular:

| | |
|---|---|
| Polyvinyl Alcohol | 1-5% |
| Stearic Acid | 10-20% |

### Manufacturing Procedure:

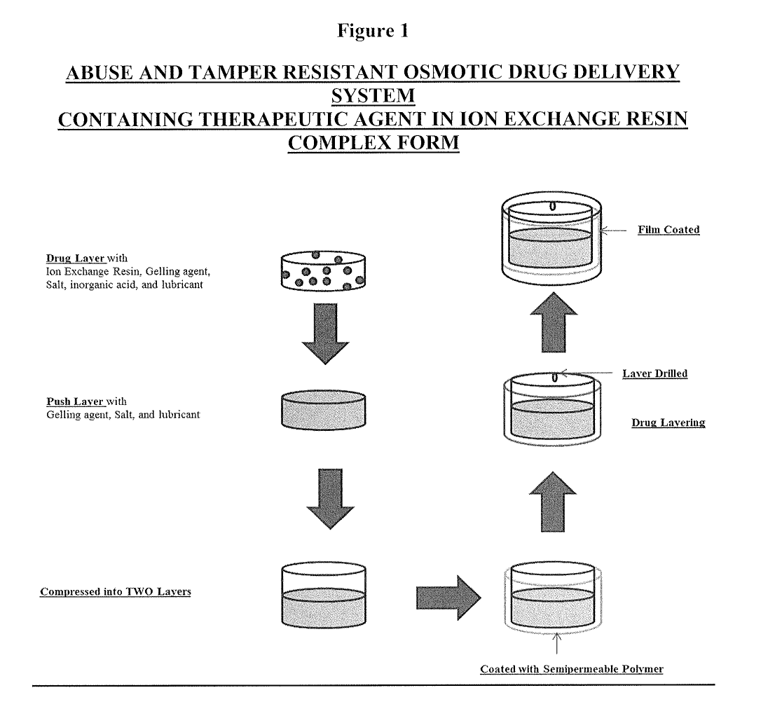

Accurately weighed and melted Precirol ATO 5 and Compritol 888 ATO mixture was added to a solution of PVP K 29/30 in ethanol kept at 50 C to form a granulating fluid used to granulate a mixture of active agent and a binder passed through #30 U.S. Standard Sieve, in a Rapid mixer granulator. The resultant granules are dried to remove moisture and sifted through 1130 U.S. Standard Sieve before blending with Polyvinyl alcohol and stearic acid mixture sifted through a #30 U.S. Standard Sieve. The resultant blend is then compressed into tablet using a suitable punch. FIG. 1. displays the release profile of such formulation in a pH 4.5 Phosphate buffer.

### FIG. 1

### Example 5. Abuse and Tamper Resistant Controlled Release Solid Dosage Form Containing Therapeutic Agent in Microencapsulated Form

Composition 11

Intragranular Part:

| | |
|---|---|
| Active | 10-60% |
| Binder | 10-40% |

Granulating Fluid:

| | |
|---|---|
| Precirol ATO | 02-30% |
| Compritol 888 ATO | 02-30% |
| PVP K 29/30 | 0-20% |
| Ethanol | q.s. |

Extragranular:

| | |
|---|---|
| Polyvinyl Alcohol | 10-50% |
| Stearic Acid | 10-20% |

### Manufacturing Procedure Followed by Heat Treatment:

Accurately weighed and melted Precirol ATO 5 and Compritol 888 ATO mixture was added to a solution of PVP K 29/30 in ethanol kept at 50 C to form a granulating fluid used to granulate a mixture of active agent and a binder passed through #30 U.S. Standard Sieve, in a Rapid mixer granulator. The resultant granules are dried to remove moisture and sifted through #30 U.S. Standard Sieve before blending with Polyvinyl alcohol and stearic acid mixture sifted through a #30 U.S. Standard Sieve. The resultant blend is then compressed into tablet using a suitable punch. The tablets will be then held at a temperature of 60° C. for a time period ranging from 5 minutes to an hour before they are set to cool down to room temperature. FIG. 1. displays the release profile of such formulation in a pH 4.5 Phosphate buffer.

### Example 6

Composition 12

Intragranular Part:

| | |
|---|---|
| Active | 10-60% |
| Binder | 10-40% |
| Ion Exchange Resin | 10-30% |

Granulating Fluid:

| | |
|---|---|
| Precirol ATO | 02-30% |
| Compritol 888 ATO | 02-30% |
| PVP K 29/30 | 0-20% |
| Ethanol | q.s. |

Extragranular:

| | |
|---|---|
| Polyvinyl Alcohol | 10-50% |
| Stearic Acid | 10-20% |

Manufacturing Procedure with Drug Complexation:

Accurately weighed and melted Precirol ATO 5 and Compritol 888 ATO mixture was added to a solution of PVP K 29/30 in ethanol kept at 50 C to form a granulating fluid used to granulate a mixture of active agent, ion exchange resin and a binder passed through #30 U.S. Standard Sieve, in a Rapid mixer granulator. The resultant granules are dried to remove moisture and sifted through #30 U.S. Standard Sieve before blending with Polyvinyl alcohol and stearic acid mixture sifted through a #30 U.S. Standard Sieve. The resultant blend is then compressed into tablet using a suitable punch.

### Example 7

Composition 13

Intragranular Part 1:

| | |
|---|---|
| Active | 10-60% |
| Binder | 10-40% |
| Ion Exchange Resin | 10-30% |

Granulating Fluid Part 1:

| | |
|---|---|
| Precirol ATO | 02-30% |
| Compritol 888 ATO | 02-30% |
| PVP K 29/30 | 0-20% |
| Ethanol | q.s. |

Extragranular Part 1:

| | |
|---|---|
| Polyvinyl Alcohol | 10-50% |
| Stearic Acid | 10-20% |

Intragranular Part 2:

| | |
|---|---|
| Polyethylene Oxide | 10-60% |
| Sodium Chloride | 10-60% |
| Hypromellose E 5 | 02-10% |
| Ferric Oxide Yellow | 01-04% |
| Butylated Hydroxy Toluene | 0-02% |

Granulating Fluid Part 2:

| | |
|---|---|
| Dehydrated Alcohol, USP | q.s. |

Extragranular Part 2:

| | |
|---|---|
| Magnesium Stearate | 01-05% |

Coating Solution:

| | |
|---|---|
| Cellulose Acetate | 05-20% |
| Acetone:Water (90:10) | q.s. |

### Manufacturing Procedure of Osmotic Controlled Release System:

Accurately weighed and melted Precirol ATO 5 and Compritol 888 ATO mixture was added to a solution of PVP K 29/30 in ethanol kept at 50 C to form a granulating fluid used to granulate a mixture of active agent, ion exchange resin and a binder passed through #30 U.S. Standard Sieve, in a Rapid mixer granulator. The resultant granules are dried to remove moisture and sifted through #30 U.S. Standard Sieve before blending with Poly(vinyl) alcohol and stearic acid mixture sifted through a #30 U.S. Standard Sieve (Layer 1). Accurately weighed quantities of Poly(ethylene) oxide, Hypromellose E 5, Ferric Oxide Yellow, Butylated Hydroxy Toluene were sifted using #30 U.S. Standard Sieve before granulating using Dehydrated alcohol. The resultant granules were then dried and sifted through #30 U.S. Standard Sieve before blending with Magnesium Stearate sifted through #30 U.S. Standard Sieve (Layer 2). The layer 1 and layer 2 blends were then transferred to a compression machine before compressing them into bi-layer tablets. The tablets were then coated using a coating solution prepared by dissolving Cellulose Acetate in acetone-water mixture.

### Example 8

Composition 14

Intragranular Part:

| | |
|---|---|
| Active | 10-60% |
| Silicified Microcrystalline Cellulose | 10-40% |

Granulating Fluid:

| | |
|---|---|
| Precirol ATO | 02-30% |
| Compritol 888 ATO | 02-30% |
| PVP K 29/30 | 0-20% |
| Ethanol | q.s. |

Extragranular:

| | |
|---|---|
| Polyvinyl Alcohol | 10-50% |
| Stearic Acid | 10-20% |

### Manufacturing Procedure Followed by Heat Treatment:

Accurately weighed and melted Precirol ATO 5 and Compritol 888 ATO mixture was added to a solution of PVP K 29/30 in ethanol kept at 50 C to form a granulating fluid used to granulate a mixture of an active agent and a binder passed through #30 U.S. Standard Sieve, in a Rapid mixer granulator. The resultant granules are dried to remove moisture and sifted through #30 U.S. Standard Sieve before blending with Polyvinyl alcohol and stearic acid mixture sifted through a #30 U.S. Standard Sieve. The resultant blend is then compressed into tablet using a suitable punch. The tablets will be then held at a temperature of 60° C. for a time period ranging from 5 minutes to an hour before they are set to cool down to room temperature.

### Example 9

Composition 15

Intragranular Part:

| | |
|---|---|
| Active | 10-60% |
| Silicified Microcrystalline Cellulose | 10-40% |
| Ion Exchange Resin | 10-30% |

Granulating Fluid:

| | |
|---|---|
| Precirol ATO | 02-30% |
| Compritol 888 ATO | 02-30% |
| PVP K 29/30 | 0-20% |
| Ethanol | q.s. |

Extragranular:

| | |
|---|---|
| Polyvinyl Alcohol | 10-50% |
| Stearic Acid | 10-20% |

Manufacturing Procedure with Drug Complexation:

Accurately weighed and melted Precirol ATO 5 and Compritol 888 ATO mixture was added to a solution of PVP K 29/30 in ethanol kept at 50 C to form a granulating fluid used to granulate a mixture of an active agent, ion exchange resin and a binder passed through #30 U.S. Standard Sieve, in a Rapid mixer granulator. The resultant granules are dried to remove moisture and sifted through #30 U.S. Standard Sieve before blending with Polyvinyl alcohol and stearic acid mixture sifted through a #30 U.S. Standard Sieve. The resultant blend is then compressed into tablet using a suitable punch.

### Example 10

Composition 16

Intragranular Part 1:

| | |
|---|---|
| Active | 10-60% |
| Silicified Microcrystalline Cellulose | 10-40% |
| Ion Exchange Resin | 10-30% |

Granulating Fluid Part 1:

| | |
|---|---|
| Precirol ATO | 02-30% |
| Compritol 888 ATO | 02-30% |
| PVP K 29/30 | 0-20% |
| Ethanol | q.s. |

Extragranular Part 1:

| | |
|---|---|
| Polyvinyl Alcohol | 10-50% |
| Stearic Acid | 10-20% |

Intragranular Part 2:

| | |
|---|---|
| Polyethylene Oxide | 10-60% |
| Sodium Chloride | 10-60% |
| Hypromellose E 5 | 02-10% |
| Ferric Oxide Yellow | 01-04% |
| Butylated Hydroxy Toluene | 0-02% |

Granulating Fluid Part 2:

| | |
|---|---|
| Dehydrated Alcohol, USP | q.s. |

Extragranular Part 2:

| | |
|---|---|
| Magnesium Stearate | 01-05% |

Coating Solution:

| | |
|---|---|
| Cellulose Acetate | 05-20% |
| Acetone:Water (90:10) | q.s. |

### Manufacturing Procedure of Osmotic Controlled Release System:

Accurately weighed and melted Precirol ATO 5 and Compritol 888 ATO mixture was added to a solution of PVP K 29/30 in ethanol kept at 50° C. to form a granulating fluid used to granulate a mixture of an active agent, ion exchange resin and a binder passed through #30 U.S. Standard Sieve, in a Rapid mixer granulator. The resultant granules are dried to remove moisture and sifted through #30 U.S. Standard Sieve before blending with Poly(vinyl) alcohol and stearic acid mixture sifted through a #30 U.S. Standard Sieve (Layer 1). Accurately weighed quantities of Poly(ethylene) oxide, Hypromellose E 5, Ferric Oxide Yellow, Butylated Hydroxy Toluene were sifted using #30 U.S. Standard Sieve before granulating using Dehydrated alcohol. The resultant granules were then dried and sifted through #30 U.S. Standard Sieve before blending with Magnesium Stearate sifted through #30 U.S. Standard Sieve (Layer 2). The layer 1 and layer 2 blends were then transferred to a compression machine before compressing them into bi-layer tablets. The tablets were then coated using a coating solution prepared by dissolving Cellulose Acetate in acetone-water mixture.

### Example #11

Composition 17

Intragranular Part:

| | |
|---|---|
| Active and/or Resinated Active | 10-60% |
| Polyethylene Oxide | 10-40% |
| PVP K 29/30 | 0-20% |
| Butylated Hydroxy Toluene | 0.01-0.5% |

Granulating Fluid:

| | |
|---|---|
| IPA | q.s. |
| Purified Water | q.s. |

Extragranular:

| | |
|---|---|
| Stearic Acid | 10-20% |

Coating 1:

| | |
|---|---|
| Polyethylene Oxide WSR N80 | 0-10% |
| PEG 6000 | 1-2% |
| Purified Water | q.s. |

Coating II:

| | |
|---|---|
| EthylCellulose | 5-10% |
| Triethyl Citrate NF | 1-4% |
| Methanol USP | 10-60% |
| Dehydrated Alcohol USP | 10-30% |

### Manufacturing Procedure:

Accurately weighed quantities of all the ingredients in the intragranular part (Active, Active-Resin Complex, Poly(ethylene) Oxide, PVP K 29/30, Butylated Hydroxy Toluene were sifted through #30 U.S. Standard sieve followed by dry mixing in a Kitchen Aid® planetary mixer for 5 minutes. Granulating fluid consisting of IPA and water mixture is used to granulate the dry mass from the intragranular part and the resultant granules are dried to remove moisture and sifted through #30 U.S. Standard Sieve. The granules are then blended with stearic acid pre-sifted using a #30 U.S. Standard Sieve before compressing using appropriate punches. The tablets are then subjected to coating using coating solution I, obtained by dissolving Poly(ethylene) oxide and Poly(ethylene) glycol mixture in purified water. The tablets are then dried before subjecting to another layer of coating using coating solution II. Coating solution II is prepared by dispersing Ethylcellulose and Triethyl citrate in ethanol to form a clear gel which is then added to methanol.

### Example 12

Composition 18

Intragranular Part:

| | |
|---|---|
| Active | 10-60% |
| Xanthum Gum | 25-50% |
| Precirol ATO | 02-30% |
| Compritol 888 ATO | 02-30% |
| Polyvinyl Alcohol | 10-50% |
| NaCl | 0-10% |

Granulating Fluid:

| | |
|---|---|
| PVP K 29/30 | 0-20% |
| Ethanol | q.s. |

Extragranular:

| | |
|---|---|
| Stearic Acid | 5-20% |

Manufacturing Procedure: Accurately weighed quantities of all the ingredients in the intragranular part (Active, Xanthum gum, Precirol ATO, Compritol 888 ATO, NaCl, PVA were sifted through #30 U.S. Standard sieve followed by dry mixing in a Kitchen Aid® 12 planetary mixer for 5 minutes. Granulating fluid consisting of PVA dissolved in ethanol is used to granulate the dry mass from the intragranular part and the resultant granules are dried to remove moisture and sifted through #30 U.S. Standard Sieve. The granules are then blended with stearic acid pre-sifted using a #30 U.S. Standard Sieve before compressing using appropriate punches.

### Example 13

Composition 19

Intragranular Part:

| | |
|---|---|
| Active | 10-60% |
| Silicified Microcrystalline Cellulose | 10-40% |

Granulating Fluid:

| | |
|---|---|
| Precirol ATO | 02-30% |
| Compritol 888 ATO | 02-30% |
| PVP K 29/30 | 0-20% |

| | | | |
|---|---|---|---|
| | | | Qty/Batch |
| S. No | Ingredients | mg/tab | (g) |

Dry Blend

| | | | |
|---|---|---|---|
| 1 | Oxycodone HCl, USP | 40.00 | 420.00 |
| 2 | Polyvinyl Alcohol | 15.00 | 150.00 |
| 3 | Xanthan Gum | 45.40 | 454.00 |
| 4 | Sodium Chloride, NF | 4.00 | 40.00 |

Granulating Fluid

| | | | |
|---|---|---|---|
| 5 | Compritol 888ATO | 20.00 | 200.00 |
| | (Glyceryl Dibehenate) | | |
| 6 | Precirol ATO | 8.10 | 81.00 |
| | (Glyceryl Distearate) | | |
| 7 | Ethanol | | |
| 8 | Povidone K 29/32 | 2.50 | 25.00 |

Extra Granular

| | | | |
|---|---|---|---|
| 9 | Stearic acid | 20.00 | 200.00 |
| | Total | 155.00 | 1550.00 |

| | |
|---|---|
| Ethanol | q.s. |

Extragranular:

| | |
|---|---|
| PEO Coagulant POLYOX | 10-60% |
| Stearic Acid | 10-20% |

### Manufacturing Procedure Followed by Heat Treatment:

Accurately weighed and melted Precirol ATO 5 and Compritol 888 ATO mixture was added to a solution of PVP K 29/30 in ethanol kept at 50° C. to form a granulating fluid used to granulate a mixture of an active agent and a binder passed through #30 U.S. Standard Sieve, in a Rapid mixer granulator. The resultant granules are dried to remove moisture and sifted through #30 U.S. Standard Sieve before blending with Poly(ethylene oxide) and stearic acid mixture sifted through a #30 U.S. Standard Sieve. The resultant blend is then compressed into tablet using a suitable punch. The tablets will be then held at a temperature of 60° C. for a time period ranging from 5 minutes to an hour before they are set to cool down to room temperature.

### Example 14

Composition 20

| | | | |
|---|---|---|---|
| | | | Qty/Batch |
| S. No | Ingredients | mg/tab | (g) |

Dry Blend

| | | | |
|---|---|---|---|
| 1 | Oxycodone HCl, USP | 40.00 | 420.00 |
| 2 | Polyvinyl Alcohol | 15.00 | 150.00 |
| 3 | Xanthan Gum | 45.40 | 454.00 |
| 4 | Sodium Chloride, NF | 4.00 | 40.00 |

Granulating Fluid

| | | | |
|---|---|---|---|
| 5 | Compritol 888ATO | 20.00 | 200.00 |
| | (Glyceryl Dibehenate) | | |
| 6 | Precirol ATO | 8.10 | 81.00 |
| | (Glyceryl Distearate) | | |
| 7 | Ethanol | | |
| 8 | Povidone K 29/32 | 2.50 | 25.00 |

Extra Granular

| | | | |
|---|---|---|---|
| 9 | Stearic acid | 20.00 | 200.00 |
| | Total | 155.00 | 1550.00 |

### Manufacturing Procedure:

1. Accurately weighed Oxycodone HCl (Item #1), Polyvinyl Alcohol (Item #2), Xanthan Gum (Item #3) and Sodium Chloride (Item #4) screened together through #30 screen.
2. Hot Melt Solvent Granulation: Melt Compritol 888 ATO (Item #5), and Precirol ATO (Item #6) together in a vessel and continue the melting until get a clear solution.
3. Dissolve Polyvinyl Pyrrolidone (PVP K 29/30) (Item #8) in hot Ethanol (heat it to 55-60° C.) (Item #3) and maintain the temperature of the solution till add to the Step #2 materials.
4. Add hot ethanol solution to molten mass Compritol and Precirol.
5. Continue heating and mixing until get a clear solution.
6. Granulate the Step #1 materials and add additional hot ethanol, if required.
7. Dry the granules at room temperature and screen through #30 mesh.
8. Add Stearic acid (Item #9) and mix for 10 minutes.
9. Compress the blend into tablets using rotary compression machine.
10. Compressed tablets melted by passing through IR tunnel.

While the invention has been described in detail and with reference to specific examples thereof, it will be apparent to one skilled in the art that various changes and modifications can be made therein without departing from the spirit and scope thereof.

What is claimed is:

1. An lipophilic abuse and tamper resistant drug delivery system comprising:
i) at least one active agent susceptible to abuse selected from the group consisting of opiates, opioids, tranquilizers, stimulants, narcotics, and combinations thereof;
ii) optionally, at least one ion exchange resin;
iii) at least one binder for granulation;
iv) optionally at least one surfactant;
v) optionally at least one wax;
vi) optionally, at least one synthetic or natural polymer;
vii) optionally at least one excipients; and
viii) optionally at least one viscosity enhancing agent.

2. A drug delivery system according to claim 1, which is in the form of a tablet.

3. A drug delivery system according to claim 1, which is in the form of a coated tablet.

4. A drug delivery system according to claim 1, which is in the form of an uncoated tablet.

5. A drug delivery system according to claim 1, which is in multiparticulate form.

6. A drug delivery system according to claim 1, wherein the at least one active agent susceptible to abuse is present in an amount of about 10 to about 60 wt % of the composition.

7. A drug delivery system according to claim 1, wherein the at least one ion exchange resin is present in an amount of about 10 to about 30 wt % of the composition.

8. A drug delivery system according to claim 1, wherein the ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof.

9. A drug delivery system according to claim 1, wherein the at least one binder for granulation is present in an amount of about 10 to about 40 wt % of the composition.

10. A drug delivery system according to claim 1, wherein the at least binder for granulation is present and is selected from the group consisting of natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, carnauba wax, glycerol behenate (COMPRITOL® 888 ATO), glycerylmonostereate, glycerol palmitostearate (PRECIROL®), and hydrophilic substances selected from a group of water soluble or water insoluble, non-gelling binders, Poly(vinyl) pyrrolidone, Poly(vinyl) alcohol, starch, corn starch, pregelatinized starch, microcrystalline cellulose (MCC), silicified MCC, microfine cellulose, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropylmethyl celluloses, and combinations thereof.

11. A drug delivery system according to claim 1, wherein the at least one surfactant is present in an amount of about 2 to about 30 wt % of the composition.

12. A drug delivery system according to claim 1, wherein the at least one wax is present in an amount of about 2 to about 30 wt % of the composition.

13. A drug delivery system according to claim 12, wherein the at least one wax is selected from the group consisting of carnauba wax, white wax, natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, carnauba wax, and combinations thereof.

14. A drug delivery system according to claim 1, wherein the at least one synthetic or natural polymer is present in an amount of about 0 to about 20 wt % of the composition.

15. A dosage form according to claim 14, at least one synthetic or natural polymer is at least one polymer selected from the group consisting of polyethylene oxide, polymethylene oxide, polypropylene oxide, polyethylene, polypropylene, polyvinyl chloride, polycarbonate, polystyrene, polyacrylate, copolymers and mixtures thereof.

16. A drug delivery system according to claim 1, wherein the at least one excipients is present in an amount of about 10 to about 50 wt % of the composition.

17. A drug delivery system according to claim 1, wherein the at least one viscosity enhancing agent is present in an amount of about 10 to about 20 wt % of the composition.

18. A dosage form according to claim 19, wherein the at least one viscosity-increasing agent is selected from the group consisting of microcrystalline cellulose with 11 wt. % carboxymethylcellulose sodium (Avice® RC 591), carboxymethylcellulose sodium (Blanose®, CMC-Na C300P®, Frimulsion BLC-5®, Tylose C300 P®), polyacrylic acid (Carbopol® 980 NF, Carbopol® 981), locust bean flour (Cesagum® LA-200, Cesagum® LID/150, Cesagum® LN-1), citrus pectin (Cesapectin® HM Medium Rapid Set), waxy maize starch (C*Gel 04201®), sodium alginate (Frimulsion ALG (E401)®), guar flour (Frimulsion BM®, Polygum 26/1-75®), iota carrageen (Frimulsion D021®), karaya gum, gellan gum (Kelcogel F®, Kelcogel LT1000), galactomannan (Meyprogat 1500), tara bean flour (Polygum 43/1®), propylene glycol alginate (Protanal-Ester SD-LB®), sodium hyaluronate, apple pectin, pectin from lemon peel, sodium hyaluronate, tragacanth, tara gum (Vidogum SP 200®), fermented polysaccharide welan gum (K1A96) and xanthan gum (Xantural 180®).

19. A dosage form according to claim 1, wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

20. A dosage form according to claim 1, wherein the dosage form has a breaking strength of at least 1000 N.

21. A dosage form according to claim 1, which comprises at least one active ingredient at least partially in controlled release form.

22. A dosage form according to claim 1, wherein the at least one active agent susceptible to abuse is present in a controlled release matrix.

23. A process for the production of a dosage form according to claim 1, comprising:
mixing components: i) at least one active agent susceptible to abuse; ii) optionally, at least one ion exchange resin; iii) at least one binder for granulation; iv) optionally at least one surfactant; v) optionally at least one wax; vi) optionally, at least one synthetic or natural polymer; vii) optionally at least one excipients; and viii) optionally at least one viscosity enhancing agent, to form a resultant mixture, and
press-forming the resultant mixture, optionally after granulation, to yield the dosage form with preceding, simultaneous, or subsequent exposure to heat.

24. A process according to claim 25, wherein granulation is performed by means of a melt process.

25. A process according to claim 25 which comprises press-forming the resultant mixture to yield a press-formed product, and exposing the press-formed product to heat to yield the dosage form.

26. A dosage form obtainable by a process according to claim 25.

27. A dosage form obtainable by a process according to claim 26.

28. A dosage form obtainable by a process according to claim 27.

29. An oral abuse and tamper resistant pharmaceutical solid dosage form comprising:
i) at least one active agent susceptible to abuse selected from the group consisting of opiates, opioids, tranquilizers, stimulants and narcotics;
ii) at least one surfactant, wherein the surfactant is selected from the group consisting of high hydrophilic/lipophilic balance (HLB) surfactants, low HLB surfactants, and a combination thereof;
iii) at least one ethylcellulose polymer, wherein the ethylcellulose polymer is selected from the group consisting of high viscosity ethylcellulose polymer, low viscosity ethylcellulose polymer, and a combination thereof;
iv) oleic acid; and
v) at least one hydrophilic solvent,
wherein said oral abuse and tamper resistant pharmaceutical solid dosage is in unit dosage form.

30. The oral abuse and tamper resistant pharmaceutical solid dosage of claim 29, wherein the at least one active agent susceptible to abuse is present in an amount of about 10 to about 60 wt % of the composition.

31. The oral abuse and tamper resistant pharmaceutical solid dosage of claim 29, wherein the at least one surfactant is present in an amount of about 2 to about 30 wt % of the composition.

32. The oral abuse and tamper resistant pharmaceutical solid dosage of claim 29, wherein the polymer is present in an amount of about 0 to about 20 wt % of the composition.

33. The oral abuse and tamper resistant pharmaceutical solid dosage of claim 29, wherein the oleic acid is present in an amount of 10 to 70 wt % of the composition.

34. The oral abuse and tamper resistant pharmaceutical solid dosage of claim 29, wherein the at least one hydrophilic solvent is present in an amount of about 5 to about 30 wt % of the composition.

35. The oral abuse and tamper resistant pharmaceutical solid dosage of claim 29, wherein the at least one hydrophilic solvent is selected from the group consisting of water, ethanol, glycerol, glycols, polyols, and combinations thereof.

36. The oral abuse and tamper resistant pharmaceutical solid dosage form of claim 29, wherein the composition is immediate release.

37. The oral abuse and tamper resistant pharmaceutical solid dosage form of claim 29, wherein the composition is delayed release.

38. The oral abuse and tamper resistant pharmaceutical solid dosage form of claim 29 wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

39. The oral abuse and tamper resistant pharmaceutical solid dosage form of claim 29, wherein the at least one active agent susceptible to abuse comprises oxymorphone or oxycodone.

40. An oral abuse and tamper resistant pharmaceutical solid dosage form comprising:
i) oxycodone present in an amount of about 7 to about 9 wt % of the composition;
ii) ethylcellulose present in an amount of about 9 to about 11 wt % of the composition;
iii) oleic acid present in an amount of about 50 to about 70 wt % of the composition;
iv) PEG-8 caprylic/capric glycerides present in an amount of about 8 to about 12 wt % of the composition; and
v) polyoxyl 40 hydrogenated castor oil present in an amount of about 8 to about 12 wt % of the composition.

41. A capsule comprising the oral abuse and tamper resistant pharmaceutical solid dosage form of claim 40.

42. The capsule of claim 40, wherein the capsule is a hard gelatin capsule or a soft gelatin capsule.

43. A method of making oral abuse and tamper resistant pharmaceutical solid dosage form comprising a tamper resistant controlled release matrix comprising:
i) at least one active agent susceptible to abuse selected from the group consisting of opiates, opioids, tranquilizers, stimulants and narcotics; ii) at least one surfactant, wherein the surfactant is selected from the group consisting of high hydrophilic/lipophilic balance (HLB) surfactants, low HLB surfactants, and a combination thereof; iii) at least one ethylcellulose polymer, wherein the ethylcellulose polymer is selected from the group consisting of high viscosity ethylcellulose polymer, low viscosity ethylcellulose polymer, and a combination thereof; iv) oleic acid; and v) at least one hydrophilic solvent,
wherein composition is in a unit dosage form, the method comprising:
mixing the surfactant, ethylcellulose polymer, and oleic acid;
adding the drug susceptible to abuse with continuous mixing;
heating; and
encapsulating,
thereby making the oral abuse and tamper resistant pharmaceutical solid dosage form.

44. The method of claim 43, wherein the at least one active agent susceptible to abuse is present in an amount of about 10 to about 60 wt % of the composition.

45. The method of claim 43, wherein the at least one surfactant is present in an amount of about 2 to about 30 wt % of the composition.

46. The method of claim 43, wherein the polymer is present in an amount of about 0 to about 20 wt % of the composition.

47. The method of claim 43, wherein the oleic acid is present in an amount of 10 to 70 wt % of the composition.

48. The method of claim 43, wherein the at least one hydrophilic solvent is present in an amount of about 5 to about 30 wt % of the composition.

49. The method of claim 43, wherein the at least one hydrophilic solvent is selected from the group consisting of water, ethanol, glycerol, glycols, polyols, and combinations thereof.

50. The method of claim 43, wherein the composition is immediate release.

51. The method of claim 43, wherein the composition is delayed release.

52. The method of claim 43 wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

53. The method of claim 43, wherein the at least one active agent susceptible to abuse comprises oxymorphone or oxycodone.

54. An oral abuse and tamper resistant pharmaceutical solid dosage form composition comprising a tamper resistant controlled release matrix, wherein the dosage form comprises:
i) at least one active agent susceptible to abuse selected from the group consisting of opiates, opioids, tranquilizers, stimulants, narcotics, and combinations thereof;
ii) at least one ion exchange resin;
iii) at least one swellable polyethylene polymer;
iv) at least one non-swellable low molecular weight polyethylene glycol; and
iv) at least one hydrophobic binder,
wherein said composition is in a unit dosage form.

55. The oral abuse and tamper resistant pharmaceutical solid dosage form of claim 54, wherein the pharmaceutical composition is in tablet form.

56. The oral abuse and tamper resistant pharmaceutical solid dosage form of claim 54, wherein the at least one active agent susceptible to abuse is present in an amount of about 10 to about 60 wt % of the composition.

57. The oral abuse and tamper resistant pharmaceutical solid dosage form of claim 54, wherein the at least one ion exchange resin is present in an amount of about 10 to about 30 wt % of the composition.

58. The oral abuse and tamper resistant pharmaceutical solid dosage form of claim 54, wherein the at least one ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof.

59. The oral abuse and tamper resistant pharmaceutical solid dosage form of claim 54, wherein the at least one swellable polyethylene polymer present in an amount of about 10 to about 60 wt % of the composition.

60. The oral abuse and tamper resistant pharmaceutical solid dosage form of claim 54, wherein the at least one non-swellable low molecular weight polyethylene glycol present in an amount of about 10 to about 60 wt % of the composition.

61. The oral abuse and tamper resistant pharmaceutical solid dosage form of claim 54, wherein the at least one hydrophobic binder is present in an amount of about 10 to about 40 wt % of the composition.

62. The oral abuse and tamper resistant pharmaceutical solid dosage form of claim 54, wherein the at least one hydrophobic binder is selected from the group consisting of natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, and carnauba wax.

63. The oral abuse and tamper resistant pharmaceutical solid dosage form of claim 54 wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

64. An oral abuse and tamper resistant pharmaceutical solid dosage form composition comprising a tamper resistant controlled release matrix, wherein the composition comprises:
i) oxycodone HCl present in an amount of about 10 to about 60 wt % of the composition;
ii) sodium polystyrene sulfonate present in an amount of about 10 to about 60 wt % of the composition;
iii) polyethylene oxide present in an amount of about 10 to about 60 wt % of the composition;
iv) polyethylene glycol present in an amount of about 10 to about 60 wt % of the composition;
v) carnuba wax present in an amount of about 10 to about 60 wt % of the composition;
vi) microcrystalline cellulose present in an amount of about 10 to about 60 wt % of the composition;
vi) colloidal silicon dioxide present in an amount of about 10 to about 60 wt % of the composition; and
vii) stearic acid present in an amount of about 10 to about 60 wt % of the composition.

65. A method of making an oral abuse and tamper resistant pharmaceutical solid dosage form comprising a tamper resistant controlled release matrix the method comprising the steps of:
providing the ingredients: i) at least one active agent susceptible to abuse selected from the group consisting of opiates, opioids, tranquilizers, stimulants, narcotics, and combinations thereof; ii) at least one ion exchange resin; iii) at least one swellable polyethylene polymer; iv) at least one non-swellable low molecular weight polyethylene glycol; and iv) at least one hydrophobic binder,
the method comprising:
mixing all ingredients;
passing through a 40 mesh screen;
compressing into tablets; and
curing at about 70° C. for about 200 seconds.

66. The method of claim 65, wherein the pharmaceutical composition is in tablet form.

67. The method of claim 65, wherein the at least one active agent susceptible to abuse is present in an amount of about 10 to about 60 wt % of the composition.

68. The method of claim 65, wherein the at least one ion exchange resin is present in an amount of about 10 to about 30 wt % of the composition.

69. The method of claim 65, wherein the at least one ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof.

70. The method of claim 65, wherein the at least one swellable polyethylene polymer present in an amount of about 10 to about 60 wt % of the composition.

71. The method of claim 65, wherein the at least one non-swellable low molecular weight polyethylene glycol present in an amount of about 10 to about 60 wt % of the composition.

72. The method of claim 65, wherein the at least one hydrophobic binder is present in an amount of about 10 to about 40 wt % of the composition.

73. The method of claim 65, wherein the at least one hydrophobic binder is selected from the group consisting of natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, and carnauba wax.

74. The method of claim 65 wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

75. An oral abuse and tamper resistant pharmaceutical solid dosage form composition comprising granules in an extragranular matrix, wherein:
the granules comprise:
i) at least one active agent susceptible to abuse selected from the group consisting of opiates, opioids, tranquilizers, stimulants, narcotics, and combinations thereof;
ii) optionally, at least one ion exchange resin;
iii) at least one binder for granulation;
iv) optionally at least one surfactant;
v) optionally at least one wax;
vi) optionally, at least one synthetic or natural polymer;
vii) optionally at least one excipients, and
the extragranular matrix comprises:
viii) at least one synthetic or natural polymer
ix) at least one gelling agent;
x) at least one osmotic agent selected from the group consisting of salts and organic acids
xi) optionally at least one viscosity enhancing agent,
wherein the pharmaceutical composition is an osmotic controlled release dosage form.

76. The oral abuse and tamper resistant pharmaceutical solid dosage form of claim 75, wherein the at least one active agent susceptible to abuse present in an amount of about 10 to about 60 wt % of the composition.

77. The oral abuse and tamper resistant pharmaceutical solid dosage form of claim 75, wherein the at least one ion exchange resin is present in an amount of about 10% to about 30% of the composition.

78. The oral abuse and tamper resistant pharmaceutical solid dosage of claim 75, wherein the ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof.

79. The oral abuse and tamper resistant pharmaceutical solid dosage form of claim 75, wherein the at least one gelling agent is present in an amount of about 10 to about 60 wt % of the composition.

80. The oral abuse and tamper resistant pharmaceutical solid dosage of claim 75, wherein the gelling agent in an effective amount to impart a viscosity unsuitable for parenteral administration when the dosage form is subjected to tampering wherein the gelling agent is selected from the group consisting of mannitol, sorbitol, starch, starch derivatives, cellulose derivatives, microcrystalline cellulose, sodium caboxymethyl cellulose, methylcellulose, ethyl cellulose, hydroxyethyl cellulose, hydroxypropyl cellulose, hydroxypropyl methylcellulose, attapulgites, bentonites, dextrins, alginates, carrageenan, gum tragacanth, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and combinations thereof.

81. The oral abuse and tamper resistant pharmaceutical solid dosage of claim 75, wherein the at least one osmotic agent selected from the group consisting of salts and organic acids, present in an amount of about 10 to about 60 wt % of the composition.

82. The oral abuse and tamper resistant pharmaceutical solid dosage form of claim 75 wherein the controlled release dosage form is a bilayer osmotic controlled release dosage form.

83. The oral abuse and tamper resistant pharmaceutical solid dosage form of claim 75 wherein said osmotic controlled release dosage form comprises a bilayer tablet comprising an orifice.

84. The oral abuse and tamper resistant pharmaceutical solid dosage form of claim 75 wherein the controlled release dosage form is a matrix controlled release dosage form.

85. The oral abuse and tamper resistant pharmaceutical solid dosage form of claim 76, wherein the at least one active agent susceptible to abuse is present in an amount of about 10 to about 60 wt % of the composition.

86. The oral abuse and tamper resistant pharmaceutical solid dosage form of claim 75, wherein the pharmaceutical composition is in unit dose form.

87. The oral abuse and tamper resistant pharmaceutical solid dosage form of claim 75, wherein the pharmaceutical composition is in tablet form.

88. The oral abuse and tamper resistant pharmaceutical solid dosage form of claim 75 wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

89. An oral abuse and tamper resistant pharmaceutical solid dosage form composition comprising a drug layer and a push layer, wherein the drug layer comprises:
i) oxycodone present in an amount of about 10 to about 60 wt % of the composition;
ii) polyethylene oxide present in an amount of about 10 to about 60 wt % of the composition;
iii) povidone present in an amount of about 10 to about 60 wt % of the composition;
iv) succinic acid present in an amount of about 10 to about 60 wt % of the composition;
v) magnesium stearate present in an amount of about 10 to about 60 wt % of the composition;
vi) butylated hydroxytoluene present in an amount of about 10 to about 60 wt % of the composition;
vii) isopropyl alcohol present in an amount of about 10 to about 60 wt % of the composition; and
viii) water, q.s.,
further wherein the push layer comprises:
i) polyethylene oxide present in an amount of about 10 to about 60 wt % of the composition;
ii) sodium chloride in an amount of about 10 to about 60 wt % of the composition;
iii) povidone present in an amount of about 10 to about 60 wt % of the composition;
iv) magnesium stearate present in an amount of about 10 to about 60 wt % of the composition;
v) butylated hydroxytoluene present in an amount of about 10 to about 60 wt % of the composition;
vi) yellow iron oxide present in an amount of about 10 to about 60 wt % of the composition;
vii) isopropyl alcohol present in an amount of about 10 to about 60 wt % of the composition; and
viii) water, q.s.

90. A method of making an oral abuse and tamper resistant pharmaceutical solid dosage form comprising granules in an extragranular matrix, the method comprising:
providing the granule ingredients which comprise: i) at least one active agent susceptible to abuse selected from the group consisting of opiates, opioids, tranquilizers, stimulants, narcotics, and combinations thereof; ii) optionally, at least one ion exchange resin; iii) at least one binder for granulation; iv) optionally at least one surfactant; v) optionally at least one wax; vi) optionally, at least one synthetic or natural polymer; vii) optionally at least one excipients,
mixing the granule ingredients;
providing the extragranular matrix ingredients which comprise: viii) at least one synthetic or natural polymer; ix) at least one gelling agent; x) at least one osmotic agent selected from the group consisting of salts and organic acids; xi) optionally at least one viscosity enhancing agent,
mixing the extragranular ingredients;
combining the granular and extragranular ingredients; and
making the osmotic controlled release dosage form.

91. The method of claim 90, wherein the at least one active agent susceptible to abuse present in an amount of about 10 to about 60 wt % of the composition.

92. The method of claim 90, wherein the at least one ion exchange resin is present in an amount of about 10% to about 30% of the composition.

93. The method of claim 90, wherein the ion exchange resin comprises ionizable groups attached to a polymer backbone where in the polymer backbone is formed by polymers selected from the group consisting of dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl, and combinations thereof.

94. The method of claim 90, wherein the at least one gelling agent is present in an amount of about 10 to about 60 wt % of the composition.

95. The method of claim 90, wherein the gelling agent in an effective amount to impart a viscosity unsuitable for parenteral administration when the dosage form is subjected to tampering wherein the gelling agent is selected from the group consisting of mannitol, sorbitol, starch, starch derivatives, cellulose derivatives, microcrystalline cellulose, sodium caboxymethyl cellulose, methylcellulose, ethyl cellulose, hydroxyethyl cellulose, hydroxypropyl cellulose, hydroxypropyl methylcellulose, attapulgites, bentonites, dextrins, alginates, carrageenan, gum tragacanth, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and combinations thereof.

96. The method of claim 90, wherein the at least one osmotic agent selected from the group consisting of salts and organic acids, present in an amount of about 10 to about 60 wt % of the composition.

97. The method of claim 90 wherein the controlled release dosage form is a bilayer osmotic controlled release dosage form.

98. The method of claim 90 wherein said osmotic controlled release dosage form comprises a bilayer tablet comprising an orifice.

99. The method of claim 90 wherein the controlled release dosage form is a matrix controlled release dosage form.

100. The method of claim 90, wherein the at least one active agent susceptible to abuse is present in an amount of about 10 to about 60 wt % of the composition.

101. The method of claim 90, wherein the pharmaceutical composition is in unit dose form.

102. The method of claim 90, wherein the pharmaceutical composition is in tablet form.

103. The method of claim 90 wherein the at least one active agent susceptible to abuse is selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

104. An oral abuse and tamper resistant solid dosage form comprising,
i) at least one active agent susceptible to abuse selected from the group consisting of opiates, opioids, tranquilizers, stimulants and narcotics present in complex form with one or more ion-exchange resin,
low molecular weight polyethylene oxide;
at least one water soluble ionic compound;
at least one non-digestible wax; and
a mixture of at least one a low melting point stearic acid and at least one low melting point palmitic acid.

105. The oral abuse and tamper resistant solid dosage form of claim 104, wherein the active ingredient is selected from the group consisting of opiates, opioid, tranquilizers, stimulants, narcotics, alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

106. The oral abuse and tamper resistant solid dosage form of claim 104, wherein the polyethylene oxide is present in an amount of about 10 to about 60 wt % of the composition.

107. The oral abuse and tamper resistant solid dosage form of claim 104, wherein the polyethylene oxide molecular weight is below 200,000 and preferably 200,000.

108. The oral abuse and tamper resistant solid dosage form of claim 104, wherein the water soluble ionic compounds are selected from the group consisting of sodium chloride, potassium chloride, and mixtures thereof.

109. The oral abuse and tamper resistant solid dosage form of claim 104, wherein the non-digestible wax material is present in an amount of about 10 to about 60 wt % of the composition.

110. The oral abuse and tamper resistant solid dosage form of claim 104, wherein the non-digestible wax material is carnauba wax, white wax, natural waxes, synthetic waxes, fatty alcohols, lauryl, myristyl, stearyl, cetyl or cetostearyl alcohol, fatty acids, fatty acid esters, fatty acid glycerides, hydrogenated fats, hydrocarbons, normal waxes, stearic acid, stearyl alcohol, beeswax, glycowax, castor wax, carnauba wax, and combinations thereof.

111. The oral abuse and tamper resistant solid dosage form of claim 104, wherein polyethylene oxide from about 5 to about 75 wt %.

112. The oral abuse and tamper resistant solid dosage form of claim 104, wherein the ionic compound is present in an amount of from about 0.5% to about 30 wt % of the composition.

113. The oral abuse and tamper resistant solid dosage form of claim 104, wherein the non-digestible wax is present in an amount of from about 2.5 to about 35 wt %.

114. A lipophilic abuse and tamper resistant drug delivery system that in its final form includes a tablet with or without a protective coat, formed by compressing granules produced by hot melt granulation of an opioid active that may or may not be complexed with an ion exchange resin, achieved through addition of binders added in solid or liquid state, either with or without suitable lubrication and viscosity enhancing agents.

115. The lipophilic abuse and tamper resistant drug delivery system of claim 114 where in the final dosage form is obtained by compressing two or more granulation mixtures where in at least one layer will include granules produced by hot melt granulation of an opioid active that may or may not be complexed with an ion exchange resin, achieved through addition of binders added in solid or liquid state.

116. The lipophilic abuse and tamper resistant drug delivery system of claim 114 wherein the release profile can be modulated by either varying several components and their levels or by modifying the method of manufacturing.

117. The lipophilic abuse and tamper resistant drug delivery system of claim 114 wherein the variation of the components to alter the drug release includes modifying the concentration of drug release retarding agents in the formulation.

118. The lipophilic abuse and tamper resistant drug delivery system of claim 114 wherein the modifications in the method of manufacturing are fashioned by including or excluding steps as follows: compression followed by heating, coating of the final product using hydrophobic materials or addition of a push layer followed by coating with a semi-permeable membrane.

119. The lipophilic abuse and tamper resistant drug delivery system of claim 114 wherein the active belongs to a group of abuse-prone opioid analgesic selected from the group consisting of alfentanil, allylprodine, alphaprodine, amphetamines, anileridine, amytal, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, eszopiclone, ethoheptazine, ethylmethylthiambutene, ethylmorphine, etonitazene, etorphine, dihydroetorphine, fentanyl, fentanyl derivatives, flunitrazepam, heroin, hydrocodone, hydrocodone bitartrate, hydromorphone, hydroxypethidine, isomethadone, ketobemidone, levorphanol, levophenacylmorphan, lofentanil, meperidine, meptazinol, metazocine, methadone, methylphenidate, metopon, morphine, morphine analogues, morphine antagonists, myrophine, narceine, nembutal, nicomorphine, norlevorphanol, normethadone, nalorphine, nalbuphene, normorphine, norpipanone, opium, oxycodone, oxycodone hydrochloride, oxymorphone, papaveretum, pentazocine, phenobarbital, phenadoxone, phenomorphan, phenazocine, phenoperidine, piminodine, piritramide, propheptazine, promedol, properidine, propoxyphene, seconal, sufentanil, tapentadol, tilidine, tramadol, zaleplon, zolpidem, pharmaceutically acceptable salts thereof, derivatives thereof, and combinations thereof.

120. The lipophilic abuse and tamper resistant drug delivery system of claim 114 wherein the ion exchange resin comprises of ionizable groups attached to a polymer backbone where in the polymer backbone is usually formed by polymers, dipropylene glycol diallyl ether, polyglycol diallyl ether, triethylene glycol divinyl ether, hydroquinone diallyl ether, tetraallyloxyethanoyl, vinyl ether, vinyl acetate, vinyl butylbenzoate, crontonic acid, polyfunctional alcohol, tetraethylene glycol diacrylate, triallylamine, trimethylolpropane diallyl ether, methylenebisacrylamide, divinylbenzene, phthalic, sulphonphthalic acid, ethylene glycol, polymethyl siloxane α,γ-hydroxypropyl or combinations thereof.

121. The lipophilic abuse and tamper resistant drug delivery system of claim 114 wherein the binder for granulation is lipophilic and may consist of a single or a group of lipophilic materials that belong to a group of fatty acid esters, Compritol 888 ATO, glycerylmonostereate, precirol, and combinations thereof.

122. The lipophilic abuse and tamper resistant drug delivery system of claim 114 wherein the binder for granulation is a mixture of lipophilic substances selected from a group of fatty acid esters, Compritol 888 ATO, glycerylmonostereate, Precirol, hydrophilic substances selected from a group of water soluble or water insoluble, non-gelling binders as Poly(vinyl) pyrrolidone, Poly(vinyl) alcohol, starch, corn starch, pregelatinized starch, microcrystalline cellulose (MCC), silicified MCC, microfine cellulose, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropylmethyl celluloses, and combinations thereof.

123. The lipophilic abuse and tamper resistant drug delivery system of claim 114 where in the binder for granulation is a mixture of substances claimed in claim 4 and is melted to achieve uniformity in blend in granulation.

124. The lipophilic abuse and tamper resistant drug delivery system of claim 114 wherein the binders can be added intragranularly or extra granularly.

125. The lipophilic abuse and tamper resistant drug delivery system of claim 114 where in the viscosity enhancing agents that are organic in nature, Poly(vinyl) pyrrolidone, Poly(vinyl) alcohol, viscosity enhancing agents that are inorganic in nature, Silicon dioxide, Bentonite, and combinations thereof.

126. The lipophilic abuse and tamper resistant drug delivery system of claim 114 wherein the lubrication agents are lipophilic and may belong to a class of fatty acids, stearic acid, mystic acid, palmitic acid, and combinations thereof.

127. The lipophilic abuse and tamper resistant drug delivery system of claim 114 wherein the lubrication agents are lipophilic and may belong to a class of fatty acid esters that include glyceride esters, glyceryl monostearate, glyceryl tribehenate, glyceryl dibehenate, and sugar esters, sorbitan monostearate and sucrose monopalmitate.

128. The lipophilic abuse and tamper resistant drug delivery system of claim 114 wherein the lubrication agents are metallic salts of fatty acids, magnesium, calcium or zinc salts of stearic acid, mystic acid, palmitic acid, and combinations thereof.

129. The lipophilic abuse and tamper resistant drug delivery system of claim 115 wherein the lubrication agents belong to a class of inorganic materials, talc, calcium silicate etc.

130. The lipophilic abuse and tamper resistant drug delivery system of claim 114 wherein the tablet is coated with either hydrophilic or lipophilic coating agents, polymeric materials, derivatives of cellulose (hydroxypropylmethyl cellulose, hydroxyethyl cellulose, hydroxypropyl cellulose, and their respective derivatives), polymers of alginic acid and its salts and derivatives, derivatives of acrylic and methacrylic acid, polymers and copolymers of said acids and/or their respective esters to further retard drug extraction or to increase gastric resistance.

131. The lipophilic abuse and tamper resistant drug delivery system of claim 114 wherein the tablet coat presented in claim 12 may consist of plasticizing materials, triethyl citrate, diethyl phthalate, diacetin, triacetin, dibutyl phthalate, dibutyl tartrate, tributyl acetate, castor oil, cetyl alcohol, cetyl stearyl alcohol, fatty acids, glycerides and triglycerides and polyoxyethylene glycols.

132. The lipophilic abuse and tamper resistant drug delivery system of claim 114, wherein the at least one active agent susceptible to abuse is present in an amount of about 10 to about 60 wt % of the composition.

133. The lipophilic abuse and tamper resistant drug delivery system of claim 114, wherein the at least one surfactant is present in an amount of about 10 to about 60 wt % of the composition.

134. The lipophilic abuse and tamper resistant drug delivery system of claim 114, wherein the polymer is present in an amount of about 10 to about 60 wt % of the composition.
